(19) United States
(12) Patent Application Publication
(10) Pub. No.: US 2011/0117095 A1
(43) Pub. Date: May 19, 2011

(54) GROWTH FACTOR HTTER36

(76) Inventors: DANIEL R. SOPPET, CENTREVILLE, VA (US); HAODONG LI, GAITHERSBURG, MD (US)

(21) Appl. No.: 12/818,854

(22) Filed: Jun. 18, 2010

Related U.S. Application Data
(63) Continuation of application No. 11/768,441, filed on Jun. 26, 2007, now abandoned.
Application No. 11/768,441 is a division of application No. 11/091,334, filed on Mar. 29, 2005, now abandoned.
(60) Provisional application No. 60/557,393, filed on Mar. 30, 2004.

(51) Int. Cl. A61K 39/395; A61P 25/00; A61P 3/04

(52) U.S. Cl. 424/135.1; 424/158.1; 424/133.1; 424/145.1; 424/139.1

(57) ABSTRACT

The present invention discloses Growth Factor HTTER36 (GDF3) polypeptides and polynucleotides encoding such polypeptides. Also provided is a procedure for producing such polypeptides by recombinant techniques and therapeutic uses of the polypeptides which include the diagnosis, prevention, and treatment of wasting disorders. Also disclosed are antagonists against such polypeptide and their therapeutic uses which include the diagnosis, prevention, and treatment of obesity and obesity-related disorders. Also disclosed are diagnostic assays for detecting altered levels of the polypeptide of the present invention and mutations in the nucleic acid sequences which encode the polypeptides of the present invention.

### CROSS REFERENCE TO RELATED APPLICATIONS

This application is a continuation of U.S. patent application Ser. No. 11/768,441 filed Jun. 26, 2007, which is a division of U.S. patent application Ser. No. 11/091,334, filed Mar. 29, 2005, now abandoned, which claims the benefit of U.S. Provisional application No. 60/557,393, filed on Mar. 30, 2004, each of which is hereby incorporated by reference.

### FIELD OF THE INVENTION

This invention relates to newly identified polynucleotides, polypeptides encoded by such polynucleotides, the use of such polynucleotides and polypeptides, as well as the production of such polynucleotides and polypeptides. The polypeptide of the present invention has been putatively identified as a human transforming growth factor. More particularly, the polypeptide of the present invention has been putatively identified as a member of the transforming growth factor Beta (TGF-β) super-family and is sometimes hereafter referred to as “HTTER36” or GDF-3. The invention also relates to inhibiting the action of such polypeptides.

This invention relates to a polynucleotide and polypeptide molecules which are structurally and functionally related to TGF-β, all of which form homo-dimers of approximately 25 kd. The TGF-β family belongs to a larger, extended super family of peptide signaling molecules that includes the Mullerian inhibiting substance (Cate, R. L. et al., Cell, 45:685-698 (1986)), decapentaplegic (Padgett, R. W. et al., Nature, 325:81-84 (1987)), bone morphogenic factors (Wozney, J. M. et al, Science, 242:1528-1534 (1988)), vg1 (Weeks, D. L. and Melton, D. A. Cell, 51:861-867 (1987)), activins (Vale, W. et al., Nature, 321:776-779 (1986)), and inhibins (Mason, A. J. et al., Nature, 318:659-663 (1985)). These factors are similar to TGF-β in overall structure, but share only approximately 25% amino acid identity with the TGF-β proteins and with each other. All of these molecules are thought to play important roles in modulating growth, development and differentiation. The protein of the present invention, PGF, retains the seven cysteine residues conserved in the C-terminal, active domain of TGF-β.

TGF-β was originally described as a factor that induced normal rat kidney fibroblasts to proliferate in soft agar in the presence of epidermal growth factor (Roberts, A. B. et al., PNAS USA, 78:5339-5343 (1981)). TGF-β has subsequently been shown to exert a number of different effects in a variety of cells. For example, TGF-βcan inhibit the differentiation of certain cells of mesodermal origin (Florini, J. R. et al., J. Biol. Chem., 261:1659-16513 (1986)), induced the differentiation of others (Seyedine, S. M. et al., PNAS USA, 82:2267-2271 (1985)), and potently inhibit proliferation of various types of epithelial cells, (Tucker, R. F., Science, 226:705-707 (1984)). This last activity has lead to the speculation that one important physiologic role for TGF-β is to maintain the repressed growth state of many types of cells. Accordingly, cells that lose the ability to respond to TGF-β are more likely to exhibit uncontrolled growth and to become tumorigenic. Indeed, certain tumors such as retinoblastomas lack detectable TGF-β receptors at their cell surface and fail to respond to TGF-β, while their normal counterparts express self-surface receptors in their growth are potently inhibited by TGF-β (Kim Chi, A. et al., Science, 240:196-198 (1988)).

More specifically, TGF-β1 stimulates the anchorage-independent growth of normal rat kidney fibroblasts (Robert et al., PNAS USA, 78:5339-5343 (1981)). Since then it has been shown to be a multi-functional regulator of cell growth and differentiation (Sporn et al., Science, 233:532-534 (1986)) being capable of such diverse effects of inhibiting the growth of several human cancer cell lines (Roberts et al., PNAS-USA, 82:119-123 (1985)), mouse keratinocytes, (Coffey et al., Cancer RES., 48:1596-1602 (1988)), and T and B lymphocytes (Kehrl et al., J. Exp. Med., 163:1037-1050 (1986)). It also inhibits early hematopoietic progenitor cell proliferation (Goey et al., J. Immunol., 143:877-880 (1989)), stimulates the induction of differentiation of rat muscle mesenchymal cells and subsequent production of cartilage-specific macro molecules (Seyedine et al., J. Biol. Chem., 262:1946-1949 (1986)), causes increased synthesis and secretion of collagen (Ignotz et al., J. Biol. Chem., 261:4337-4345 (1986)), stimulates bone formation (Noda at al., Endocrinology, 124:2991-2995 (1989)), and accelerates the healing of incision wounds (Mustoe et al., Science, 237:1333-1335 (1987)).

Further, TGF-β1 stimulates formation of extracellular matrix molecules in the liver and lung. When levels of TGF-β1 are higher than normal, formation of fiber occurs in the extracellular matrix of the liver and lung, which can be fatal. High levels of TGF-β1 occur due to chemotherapy and bone marrow transplant as an attempt to treat cancers, e.g. breast cancer.

A second protein termed TGF-β2 was isolated from several sources including demineralized bone, a human prostatic adenocarcinoma cell line (Ikeda et al., Bio. Chem., 26:2406-2410 (1987)). TGF-β2 shared several functional similarities with TGF-β1. These proteins are now known to be members of a family of related growth modulatory proteins including TGF-β3 (Ten-Dijke et al., PNAS, USA, 85:471-4719 (1988)), Muellerian inhibitory substance and the inhibins. The polypeptide of the present invention has been putatively identified as a member of this family of related growth modulatory proteins.

### BACKGROUND OF THE INVENTION

Many diseases and disorders have a need for weight loss. Weight gain is a common problem associated with excessive appetite, obesity, diabetes-related obesity, metabolic syndrome (insulin resistance, alterations in glucose and lipid metabolism, increased blood pressure and visceral obesity), menopausal associated weight gain, excessive pregnancy weight gain, mental and psychological disorders such as bipolar disorder, depression, or schizophrenia, weight gain associated with the use of alterations in SNS effects on metabolism, high leptin levels in adolescent females, low perinatal birth weight (leading to childhood morbidity, such as diabetes), and changes in blood pressure such as increased blood pressure and increased incidence of hypertension. In addition, a diet high in fat exacerbates these problems.

Hepatic steatosis, or accumulation of fat in the liver, is also a problem exacerbated by a high fat diet. In rodents, hepatic steatosis induced by high fat diet is disproportionately mild compared to body fat accumulation. (R. H. Unger and L. Orci, FASEB J., 15:312-321 (2001)). Only leptin deficient ob/ob mice or leptin unresponsive (db/db, fa/fa) rats develop severe hepatic steatosis with diet of fat content as low as 6%. Reconstitution of leptin signaling in ob/ob and fa/fa animals led to rapid and dramatic decrease in hepatosteatosis. (Leclercq, I. at al., J. Gastroenterol. Hepatol., 13 (Suppl):188A (1998); and Chitturi, S. et al., Hepatology, 36:403-409 (2002)).

Ingestion of a diet high in fat does not alone result in hepatic steatosis. In humans, hepatic steatosis is mostly caused by alcoholism. The underlying conditions and pathogenesis for non-alcoholic hepatic steatosis remains unclear. Extreme obesity, uncontrolled diabetes/insulin resistance, hyperlipidemia, steroid use, or even acute starvation, rapid weight loss, and intestinal bypass are among the risk factors that favor lipogenesis in the liver and lead to steatosis.

Weight reduction, especially reduction of percent body fat, is also strongly desired outside of the medical industry. Perfecting personal body image is a goal for the weight and fitness-training industry, sports industry, and the general public. Reducing weight, specifically reducing percent body fat, is strongly desired and sought after by people of all ages, health, and sex across the United States. There is constantly a call both in the art and among the general public for additional treatments to reduce weight and/or prevent weight gain, specifically to reduce percent body fat.

Cytokines that act on adipose tissue and regulate adiposity are of intense interest as possible compositions for the treatment of weight gain associated conditions, such as obesity. Insulin, leptin, glucagon, TNF-α, 1L-6, GLP-1, growth hormone, and several other cachectic factors are known to be involved either positively or negatively in the regulation of adiposity. (ED. Rosen, Ann. N.Y. Acad. Sci., 979:143-158, discussion 188-196 (2002); MacDonald, O. A. et al., Trends Endocrinol. Metab., 13:5-11 (2002); E. D. Rosen and B. M. Spiegelman, Annu. Rev. Cell Dev. Biol., 16:145-171 (2000); and Fruhbeck, G. et al., Am. J. Physiol. Endocrinol. Metab., 280:E827-847 (2001)).

Several TGF-β superfamily members have been shown to have potent effects on adipocytes and adipose tissues. For example, TGF-β blocks adipocyte differentiation in vitro. Transgenic overexpression of TGF-β in vivo leads to lipodystrophy-like syndrome. (Petruschke, T. et al., Int. J. Obes. Relat Metab. Disord., 18:532-536 (1994); and Clouthier, D. E. et al., J. Clin. Invest, 100:2697-2713 (1997)). In addition, members of the BMP/GDF subfamily of TGF-β proteins have also been shown to have potent effects on adiposity. For example, systemic administration of GDF-8/myostatin resulted in near-total loss of white adipose tissue in addition to profound muscle wasting. (Zimmers, T. A. et al., Science, 296:1486-1488 (2002)). Conversely, GDF-8 knockout mice had defective adipogenesis and suppressed fat accumulation. (A. C. McPherron and S. J. Lee, J. Clin. Invest., 109:595-601 (2002)).

Peroxisome proliferator activated receptor (PPARγ) has been identified as a master regulator of adipocyte differentiation, adipogenesis, glucose homeostasis and lipid metabolism. (G. J. Etgen, and N. Mantlo, Curr. Top. Med. Chem., 3:1649-1661 (2003); B. M. Spiegelman, Diabetes, 47:507-514 (1998); and Spiegelman, B. M. et al., Biochimie, 79:111-112 (1997)). PPARγ is predominantly expressed in mature adipocytes and its expression is induced during preadipocyte differentiation. (Braissant, O. et al., Endocrinology, 137:354-366 (1996); Vidal-Puig, A. et al., J. Clin Invest, 97:2553-2561 (1996); and Chawla, A. et al., Endocrinology, 135:798-800 (1994)). PPARγ regulates genes central to lipid metabolism and storage, for example, acetyl-CoA synthase, aP2, phosphaenol pyruvate carboxykinase, fatty acid transport protein, and lipoprotein lipase. Non-adipocytes can be converted into adipocytes by forced PPARγ expression. (Tontonoz, P. et al., Cell, 79:1147-1156 (1994); and Hu, E. et al., Proc Natl Acad Sci USA, 92:9856-9860 (1995)). Genetic knockout mice are completely devoid of adipose tissue. (Kubota, N. et al., Mol Cell, 4:597-609 (1999); and Miles, P. D. et al., J Clin Invest, 105:287-292 (2000)). In contrast, constitutively active PPARγ mutations in human lead to increased adipocyte differentiation and obesity. (Ristow, M. et al., N Engl J Med, 339:953-959 (1998)).

In contrast to diseases and conditions associated with weight gain, many other diseases and disease-treatment regimes result in patient wasting. In some diseases, weight loss is so severe as to reduce patient survival time. patient quality of life, and may lead to death. In many instances mechanism of the severe weight loss or wasting is still unknown, making treatment difficult. For example, in human immunodeficiency virus (HIV) patient wasting is a major complication, especially in the advanced stages of the disease such as the onset of AIDS. Commonly known as AIDS wasting syndrome (AWS), the loss of body cell mass (BCM) or lean body mass (LBM) in HIV/AIDS patients is the result of anorexia, malabsorbtion and malnutrition, diarrhea, and/or altered metabolic states. Nemecheck, et al. Mayo Clin Proc, 75(4):386-94 (2000). Loss of BCM causes dire patient prognosis due to a loss of food energy and due to reduced physical functioning, fat and lean muscle tissue wasting, poor quality of life, and ultimately a significantly decreased chance of patient survival.

Cancer patients also suffer from wasting, or cachexia, which typically occurs during the final stages of cancer. Cachexia frequently occurs as an adverse reaction to cancer treatment regiems of radiation and chemotherapy which result in painful ulcers throughout the mucosal lining of the upper GI tract. This makes food and nutrient consumption difficult if not impossible for patients and they are unable to maintain normal body weight due to the decreased intake of nutrients and increased cancer metabolism. The onset of cachexia is strongly indicative of a decreased chance of cancer patient survival.

Geriatric wasting syndrome (GWS) is another disorder associated with severe weight loss. GWS affects the elderly and is characterized by a generalized loss of appetite, usually accompanied by mental, cognitive, and/or psychological disorders, such as depression, and an overall decline in the patient's quality of life. Geriatric cachexia can also be associated with infection, ulcers, and even death. Even a modest decline in body weight of an elderly patient is indicative of an increased risk of mortality. Newman, et al. J Am Geriatr Soc, 49(10):130-18 (2001).

General loss of appetite or eating disorders such as anorexia nervosa and bulimia are also associated with a severe loss of body weight. While usually accompanied by mental and psychological disorders and thereby requiring associated therapies, there is a need to increase body weight to prevent patient death.

One of the concerns of wasting or cachexia is the loss of lean body mass (LBM) due to accelerated protein breakdown and decreased protein synthesis. However, the loss of fatty tissue is also a concern for a variety of reasons such as drastically reducing a patient's total body weight and a redistribution of patient body fat. With muscle and fatty tissue reserves depleated, patients can have difficulty sustaining normal body temperature and maintaining immune defenses. Attempted, current, and potential treatment regimes, not including therapies to increase skeletal muscle growth, or lean muscle mass, attempt to increase weight and body fat in patients suffering from AWS, cancer, and GWS.

Therapies for AWS include the use of recombinant growth hormone (Schambelan, et al. Ann Intern Med, 125(11):873-82 (1996)), administration of insulin (Kabadi, et al. AIDS Patient Care, 14(11):575-9 (2000)), magestrol acetate in a multi-drug regime (will also increase lean body tissue) (Farrar, D. J., AIDS Patient Care, 13(3):149-52 (1999)), and treatment with indinavir (Carbonnel, et al., AIDS, 12(14):1777-84 (1998). Cancer wasting therapies include the use of inflammatory cytokines (Tohgo, et al., Expert Rev Anticancer Ther, 2(1):121-9 (2002)), appetite stimulation through antiserotonergic drugs, gastroprokinetic agents, branched-chain amino acids, eicosapentanoic acid, cannabinoids, melatonin, and thalidomide (Inui, A., CA Cancer. J Clin, 52(2):72-91 (2002)). Therapies for GWS include the use of progestational agents, cyproheptadines, pentoxifylline, and thalidomide to regulate proinflammatory cytokines (Yeh, et al. Am J Clin Nut, 70(2):183-97 (1999)). Despite the current technologies, however, there is still a strong need in the art for an effective treatment therapy for wasting disorders to increase patient body weight, specifically to increase fatty tissue. Ideally, new treatments will be useful in multidrug treatment in order to target replacement of both lean and fatty tissue, without interfering in disease treatment regimes.

Low maternal weight and low fetal weight are also associated with severe weight loss and can have life-long consequences as a result. About 4-7 percent of the infants born in the US suffer from low fetal weight, also called Intrauterine Growth Retardation/Restriction (IUGR) and Fetal Growth Retardation (FGR). Low fetal weight can be associated with premature or full-term fetal birth. While there is no consensus on the criteria of classification for low fetal weight, the criteria lingers between 5-10 percent of the predicted fetal weight for gastrointestinal age. Vandenbosche and Kirchner, Intrauterine Growth Retardation, Amer Acad Fam Phy, Oct. 15, 1998. Infants born with IUGR have a 6-10 times increase in the chance of perinatal mortality. In fact, low infant birth weight is the signal most important factor affecting neonatal mortality. Even if the infant survives, there is an increased chance infant morbidity due to difficulty in maintaining normal body temperature and fighting infection, and there is a good chance of the morbidity extending into childhood, and even lasting into adulthood.

Many factors are associated with IUGR, and they are divided into two (2) categories: fetoplacental factors and maternal factors. One major factor in low fetal weight is low maternal weight during pregnancy, especially up to 40 weeks of gestation. Vandenbosche and Kirchner, Intrauterine Growth Retardation, Amer Mad Fam Phy, Oct. 15, 1998. Therapies for IUGR include prenatal management, daily low-dose aspirin consumption, labor and delivery management, and management or increase of maternal body weight. Vandenbosche and Kirchner, Intrauterine Growth Retardation, Amer Acad Fain Phy, Oct. 15, 1998.

While not disease-related, the sports industry also has a call for weight gain, or more specifically, the retention of energy storing carbohydrates. Sumo wrestling requires athletes to gain and maintain high weight levels, including fatty tissue. Also, endurance sports such as running, biking, hiking, swimming, mountain and ice climbing as well as other extreme and/or endurance sports require athletes to call on energy reserves. Therefore, the ability to increase appetite and/or retain carbohydrates during extreme physical exertion could enhance performance and preserve normal body temperature in extremely cold conditions. Similarly, the military personelle could benefit during times of war, limited and extended missions, and extreme training by increasing energy reserves prior to missions.

### SUMMARY OF THE INVENTION

In accordance with one aspect of the present invention, there are provided novel mature polypeptides, as well as biologically active and diagnostically or therapeutically useful fragments, analogs and derivatives thereof. The polypeptides of the present invention are of human origin.

In accordance with another aspect of the present invention, there are provided isolated nucleic acid molecules encoding the polypeptides of the present invention, including mRNAs, cDNAs, genomic DNAs as well as analogs and biologically active and diagnostically or therapeutically useful fragments thereof.

In accordance with another aspect of the present invention there is provided an isolated nucleic acid molecule encoding a mature polypeptide expressed by the human cDNA contained in ATCC Deposit No. 97349.

In accordance with yet a further aspect of the present invention, there are provided processes for producing such polypeptide by recombinant techniques comprising culturing recombinant prokaryotic and/or eukaryotic host cells, containing a nucleic acid sequence encoding a polypeptide of the present invention.

In accordance with yet a further aspect of the present invention, there are provided processes for utilizing such polypeptides, or polynucleotides encoding such polypeptides for therapeutic purposes, for example, to stimulate appetite and/or weight gain and to increase fat content in adults and in pre- and post-natal infants, especially under high fat conditions.

In accordance with yet a further aspect of the present invention, there is also provided nucleic acid probes comprising nucleic acid molecules of sufficient length to specifically hybridize to nucleic acid sequences of the present invention.

In accordance with yet a further aspect of the present invention, there are provided antibodies against such polypeptides which may be used to inhibit the action of such polypeptides. Antibodies of against the polypeptides of the invention may be utilized for example, in the treatment of obesity, to stimulate weight loss, or to reduce excessive appetite.

In accordance with yet a further aspect of the present invention, there are provided agonists to the polypeptide of the present invention.

In accordance with yet another aspect of the present invention, there are provided antagonists to such polypeptides, which may be used to inhibit the action of such polypeptides, for example, in the treatment of obesity, to stimulate weight loss, or to reduce excessive appetite.

In accordance with still another aspect of the present invention, there are provided diagnostic assays for detecting diseases related to over expression of the polypeptide of the present invention and mutations in the nucleic acid sequences encoding such polypeptide.

In accordance with yet a further aspect of the present invention, there is provided a process for utilizing such polypeptides, or polynucleotides encoding such polypeptides, for in vitro purposes related to scientific research, synthesis of DNA and manufacture of DNA vectors.

These and other aspects of the present invention should be apparent to those skilled in the art from the teachings herein.

### BRIEF DESCRIPTION OF THE DRAWINGS

The following drawings are illustrative of embodiments of the invention and are not meant to limit the scope of the invention as encompassed by the claims.

FIGS. 1A-B depict the cDNA sequence and corresponding deduced amino acid sequence of HTTER36. The standard one-letter abbreviations for amino acids are used. The putative signal sequence has been underlined.

FIG. 2 is an illustration of comparative amino acid sequence homology between HTTER36 (top line) and Mus musculus putative transforming growth factor-beta, “GDF-3” (SEQ ID NO:9).

FIGS. 3A-C depict the body weight gain in adenovirus-transduced mice expressing HTTER36 (GDF3) under high fat or normal diet conditions compared to mice expressing the negative control (β-galactosidase gene). FIG. 3A depicts the body weight growth curves of the mice in each experimental group over a period of 45 days. n=4 for each treatment group. Error bars represent standard deviations. FIG. 3B depicts the body weight gains of each experimental group 45 days into the experiment as normalized by respective initial body weights. FIG. 3C depicts the percentage of epididymal fat pad (eWAT) weight by the total body weight for each experimental group. Error bars are standard errors (n=4).

FIGS. 4A-C depict the anatomical effects of adenovirus expression of HTTER36 (GDF3) in mice under high fat or normal diet conditions compared to mice expressing the adenovirus-induced β-galactosidase gene. FIGS. 4A and B depict the anatomical effect on mice from each experimental group by visual top view and total body X-ray imaging, respectively. FIG. 4C depicts the distribution of abdominal fat deposits in mice from each of the experimental groups.

FIGS. 5A-D depict the histological degrees of adipocyte hypertrophy in adenovirus-transduced mice expressing HTTER36 (GDF3) under high fat or normal diet conditions compared to mice expressing the β-galactosidase gene. The degrees of adipocyte hypertrophies were compared in terms of both cell volume size (rHᵥ) and cell mass (rHₘ) using the mice which were transduced with an adenovirus containing the β-galactosidase gene and which received the normal diet as a control.

FIGS. 5E-H depict the histological degrees of steatosis development in the liver lobules of adenovirus-transduced mice expressing HTTER36 (GDF3) under high fat or normal diet conditions compared to mice expressing the β-galactosidase gene.

FIG. 6A depicts the serum leptin levels in adenovirus-transduced mice expressing HTTER36 (GDF3) under high fat or normal diet conditions compared to mice expressing the β-galactosidase gene.

FIG. 6B depicts the serum insulin levels in adenovirus-transduced mice expressing HTTER36 (GDF3) under high fat or normal diet conditions compared to mice expressing the β-galactosidase gene.

FIGS. 6C-D depicts the blood glucose clearance in adenovirus-transduced mice expressing HTTER36 (GDF3) under high fat or normal diet conditions compared to mice expressing the β-galactosidase gene at day 5 and day 45. Blood glucose levels were measured after each experimental group was subjected to short-term and long-term diet treatment, to overnight fasting, and to an oral challenge with 2 g/kg Dextrose.

FIG. 7 depicts a Taqman RT-PCR analysis of PPARγ RNA inducted by 500 ng/mL HTTER36 (GDF-3) in human primary preadipocytes, human adipocytes, 3T3L1 cells and differentiated 3T3L1 cells. PPARγ levels are represented as the expression ratio over 18s RNA.

### DETAILED DESCRIPTION OF THE INVENTION

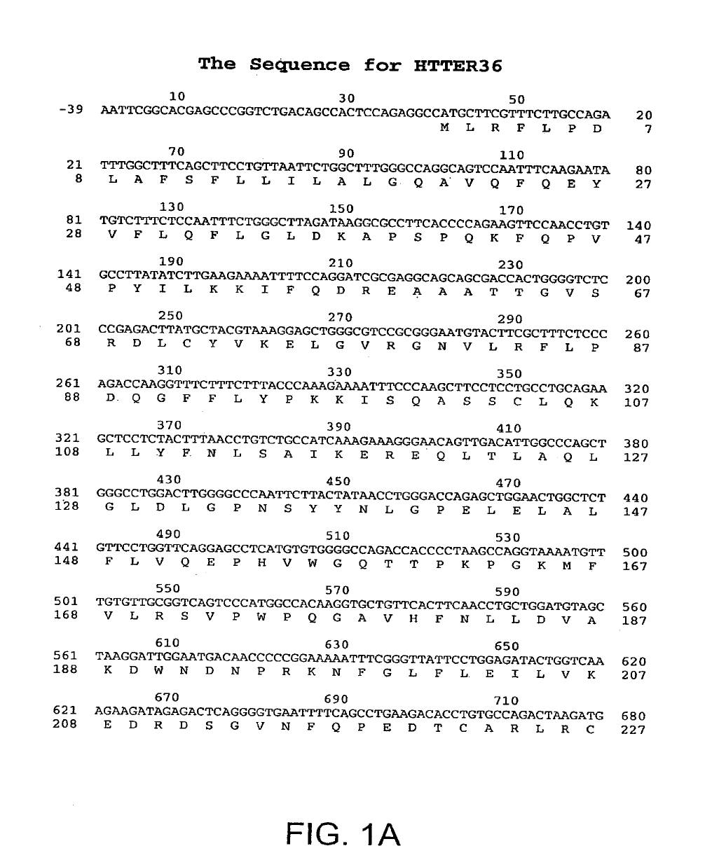

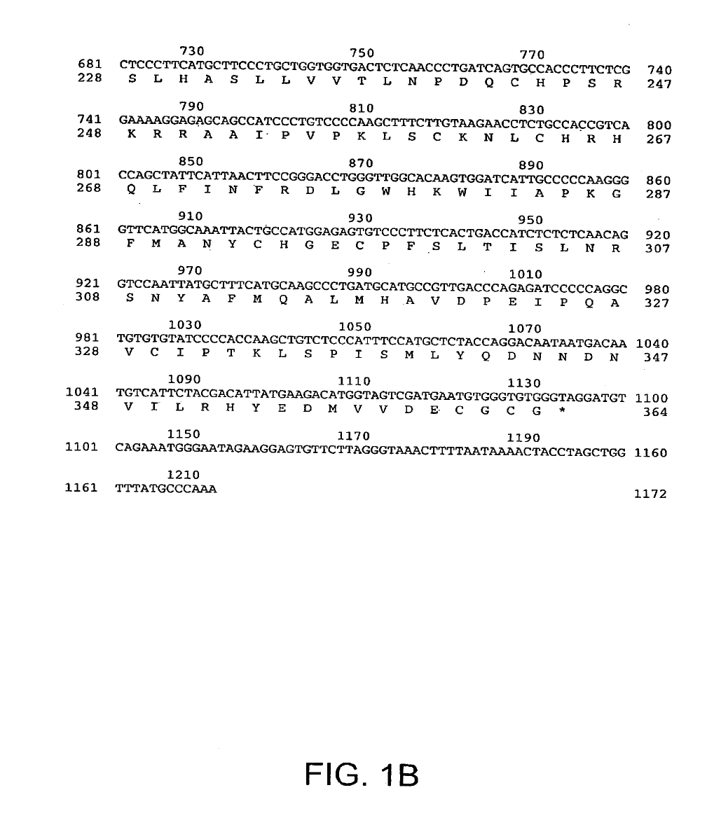

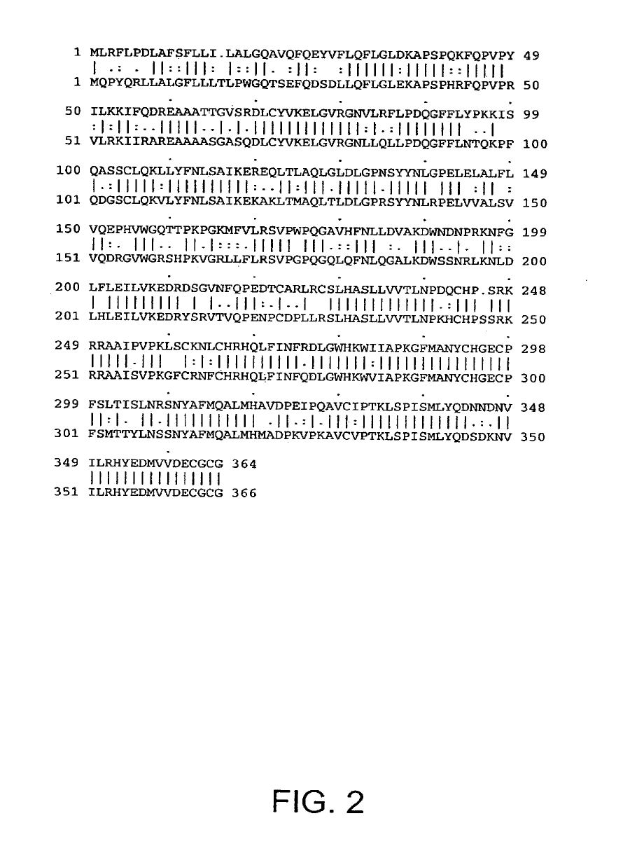

In accordance with an aspect of the present invention, there is provided an isolated nucleic acid (polynucleotide) which encodes for the mature polypeptide having the deduced amino acid sequence of FIGS. 1A-B (SEQ ID NO:2).

The polynucleotide of this invention was discovered in a human testes tumor cDNA library. It is structurally related to the TGFβ gene super-family. It contains an open reading frame encoding a polypeptide of 364 amino acids, of which the first 16 amino acids are a putative leader sequence, the next 234 amino acids are a pro-sequence and the last 114 amino acids are the active region. HTTER36 (GDF3) exhibits the highest degree of homology at the amino acid level to GDF-3 with 69% identity and 80% similarity.

Expression of HTTER36 (GDF-3) mRNA has been observed in human kidney tissue.

The first 16 amino acids represent a putative transmembrane portion which is thought to be necessary to direct the polypeptide to particular target locations for the carrying out of biological functions as hereinafter described. The transmembrane portion may also be cleaved from the polypeptide.

In accordance with another aspect of the present invention there are provided isolated polynucleotides encoding a mature polypeptide expressed by the human cDNA contained in ATCC Deposit No. 97349, deposited with the American Type Culture Collection, 10801 University Boulevard, Manassas, Va. 20110-2209, USA, on Nov. 29, 1995. The deposited material is a pBluescript SK(+) plasmid that contains the full-length HTTER36 cDNA, referred to as “PF230” when deposited.

The deposit has been made under the terms of the Budapest Treaty on the International Recognition of the Deposit of Micro-organisms for purposes of Patent Procedure. The strain will be irrevocably and without restriction or condition released to the public upon the issuance of a patent. These deposits are provided merely as convenience to those of skill in the art and are not an admission that a deposit is required under 35 U.S.C. §112. The sequence of the polynucleotides contained in the deposited materials, as well as the amino acid sequence of the polypeptides encoded thereby, are controlling in the event of any conflict with any description of sequences herein. A license may be required to make, use or sell the deposited materials, and no such license is hereby granted. References to “polynucleotides” throughout this specification includes the DNA of the deposit referred to above.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIGS. 1A-B (SEQ ID NO:1) or may be a different coding sequence which coding sequence, as a result of the redundancy or degeneracy of the genetic code, encodes the same mature polypeptide as the DNA of FIGS. 1A-B (SEQ ID NO:1).

The polynucleotide which encodes for the mature polypeptide of FIGS. 1A-B (SEQ ID NO:2) may include, but is not limited to: only the coding sequence for the mature polypeptide; the coding sequence for the mature polypeptide and additional coding sequence such as a leader or secretory sequence or a proprotein sequence; the coding sequence for the mature polypeptide (and optionally additional coding sequence) and non-coding sequence, such as introns or non-coding sequence 5′ and/or 3′ of the coding sequence for the mature polypeptide.

Thus, the term “polynucleotide encoding a polypeptide” encompasses a polynucleotide that includes only coding sequence for the polypeptide as well as a polynucleotide that includes additional coding and/or non-coding sequence.

The present invention further relates to variants of the hereinabove described polynucleotides which encode for fragments, analogs and derivatives of the polypeptide having the deduced amino acid sequence of FIGS. 1A-B (SEQ ID NO:2). The variant of the polynucleotide may be a naturally occurring allelic variant of the polynucleotide or a non-naturally occurring variant of the polynucleotide.

Particularly preferred variants include the following: 254-364; 255-364; 256-364; 257 364; 258-364; 259-364; 260-364; and 261-364. These variants would be expected to maintain HTTER36 (GDF-3) activity because they all include the cystine at position 261, which is believed to be required for the structural integrity of GDF-3. Polynucleotides encoding such polypeptides are also provided.

Thus, the present invention includes polynucleotides encoding the same mature polypeptide as shown in FIGS. 1A-B (SEQ ID NO:2) as well as variants of such polynucleotides which variants encode for a fragment, derivative or analog of the polypeptide of FIGS. 1A-B (SEQ ID NO:2). Such nucleotide variants include deletion variants, substitution variants and addition or insertion variants.

As hereinabove indicated, the polynucleotide may have a coding sequence which is a naturally occurring allelic variant of the coding sequence shown in FIGS. 1A-B (SEQ ID NO:1). As known in the art, an allelic variant is an alternate form of a polynucleotide sequence, which may have a substitution, deletion, or addition of one or more nucleotides, which does not substantially alter the function of the encoded polypeptide.

The present invention also includes polynucleotides, wherein the coding sequence for the mature polypeptide may be fused in the same reading frame to a polynucleotide sequence which aids in expression and secretion of a polypeptide from a host cell, for example, a leader sequence which functions as a secretory sequence for controlling transport of a polypeptide from the cell. The polypeptide having a leader sequence is a preprotein and may have the leader sequence cleaved by the host cell to form the mature form of the polypeptide. The polynucleotides may also encode for a proprotein which is the mature protein plus additional 5′ amino acid residues. A mature protein having a prosequence is a proprotein and is an inactive form of the protein. Once the prosequence is cleaved an active mature protein remains. Thus, for example, the polynucleotide of the present invention may encode for a mature protein, or for a protein having a prosequence or for a protein having both a prosequence and a presequence (leader sequence).

The polynucleotides of the present invention may also have the coding sequence fused in frame to a marker sequence that allows for purification of the polypeptide of the present invention. The marker sequence may be a hexa-histidine tag supplied by a pQE vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The term “gene” means the segment of DNA involved in producing a polypeptide chain; it includes regions preceding and following the coding region (leader and trailer) as well as intervening sequences (introns) between individual coding segments (exons).

Fragments of the full length HTTER36 (GDF3) gene may be used as a hybridization probe for a cDNA library to isolate the full-length gene and to isolate other genes that have a high sequence similarity to the gene or similar biological activity. Probes of this type preferably have at least 15 bases, more preferably at least 30 bases and even more preferably may contain, for example, at least 50 or more bases. The probe may also be used to identify a cDNA clone corresponding to a full-length transcript and a genomic clone or clones that contain the complete HTTER36 (GDF3) gene including regulatory and promotor regions, exons, and introns. An example of a screen comprises isolating the coding region of the gene by using the known DNA sequence to synthesize an oligonucleotide probe. Labeled oligonucleotides having a sequence complementary to that of the gene of the present invention are used to screen a library of human cDNA, genomic DNA or mRNA to determine which members of the library the probe hybridizes to.

The present invention further relates to polynucleotides that hybridize to the hereinabove-described sequences if there is at least 70%, preferably at least 90%, and more preferably at least 95% identity between the sequences. The present invention particularly relates to polynucleotides that hybridize under stringent conditions to the hereinabove-described polynucleotides. As herein used, the term “stringent conditions” means hybridization will occur only if there is at least 95% and preferably at least 97% identity between the sequences. The polynucleotides which hybridize to the hereinabove described polynucleotides in a preferred embodiment encode polypeptides which either retain substantially the same biological function or activity as the mature polypeptide encoded by the cDNA of FIGS. 1A-B (SEQ ID NO:1).

Alternatively, the polynucleotide may have at least 15 bases, preferably at least 30 bases, and more preferably at least 50 bases which hybridize to a polynucleotide of the present invention and which has an identity thereto, as hereinabove described, and which may or may not retain activity. For example, such polynucleotides may be employed as probes for the polynucleotide of SEQ ID NO:1, for example, for recovery of the polynucleotide or as a diagnostic probe or as a PCR primer.

Thus, the present invention is directed to polynucleotides having at least a 70% identity, preferably at least 90% and more preferably at least a 95% identity to a polynucleotide which encodes the polypeptide of SEQ ID NO:2 and polynucleotides complementary thereto as well as portions thereof, which portions have at least 15 consecutive or preferably at least 30 consecutive bases and most preferably at least 50 consecutive bases and to polypeptides encoded by such polynucleotides.

The present invention further relates to a polypeptide that has the deduced amino acid sequence of FIGS. 1A-B (SEQ ID NO:2), as well as fragments, analogs and derivatives of such polypeptide.

The terms “fragment,” “derivative” and “analog” when referring to the polypeptide of FIGS. 1A-B (SEQ ID NO:2), means a polypeptide that retains essentially the same biological function or activity as such polypeptide. Thus, an analog includes a proprotein that can be activated by cleavage of the proprotein portion to produce an active mature polypeptide.

The polypeptide of the present invention may be a recombinant polypeptide, a natural polypeptide or a synthetic polypeptide, preferably a recombinant polypeptide.

The fragment, derivative or analog of the polypeptide of FIGS. 1A-B (SEQ ID NO:2) may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as a leader or secretory sequence or a sequence which is employed for purification of the mature polypeptide or a proprotein sequence. Such fragments, derivatives and analogs are deemed to be within the scope of those skilled in the art from the teachings herein.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polypeptides of the present invention include the polypeptide of SEQ ID NO:2 (in particular the mature polypeptide) as well as polypeptides which have at least 70% similarity (preferably at least 70% identity) to the polypeptide of SEQ ID NO:2 and more preferably at least 90% similarity (more preferably at least 90% identity) to the polypeptide of SEQ ID NO:2 and still more preferably at least 95% similarity (still more preferably at least 95% identity) to the polypeptide of SEQ ID NO:2 and also include portions of such polypeptides with such portion of the polypeptide generally containing at least 30 amino acids and more preferably at least 50 amino acids.

As known in the art “similarity” between two polypeptides is determined by comparing the amino acid sequence and its conserved amino acid substitutes of one polypeptide to the sequence of a second polypeptide.

Fragments or portions of the polypeptides of the present invention may be employed for producing the corresponding full-length polypeptide by peptide synthesis; therefore, the fragments may be employed as intermediates for producing the full-length polypeptides. Fragments or portions of the polynucleotides of the present invention may be used to synthesize full-length polynucleotides of the present invention.

The present invention also relates to vectors that include polynucleotides of the present invention, host cells which are genetically-engineered with vectors of the invention and the production of polypeptides of the invention by recombinant techniques.

Host cells are genetically engineered (transduced or transformed or transfected) with the vectors of this invention, which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the genes of the present invention. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein. As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. In a particular embodiment of the invention, adenoviral strains are contemplated for use with the polypeptides and polynucleotides of the instant invention. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptc99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lad, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell, or the host cell can be a virus, such as an adenovirus. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)).

Moreover, introduction of the construct into a host cell, such as an adenovirus, can be mediated through the use of a shuttle vector system. In a particular embodiment, the polynucleotides of the instant invention can be ligated into a shuttle vector which can then be grafted into the adenoviral DNA. Many shuttle vector systems are available commercially. Thus, in a further embodiment, the present invention relates to the use of an adenoviral expression system kit, such as the Adeno-X Expression System kit (BD Clonetech, Ca.) in the introduction of the above-described contructs into a viral host cell.

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference. Appropriate cloning and expression vectors for use with viral hosts are described by Okada, et al., “Efficient directional cloning of recombinant adenovirus vectors using DNA-protein complex.” Nucleic Acids Res 26(8):1947-50 (1998).

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM1 (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well known to those skilled in the art.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The polypeptides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic, eukaryotic, or viral host (for example, by bacterial, yeast, higher plant, insect, viral, and mammalian cells in culture). Depending upon the host employed in a recombinant production procedure, the polypeptides of the present invention may be glycosylated or may be non-glycosylated. Polypeptides of the invention may also include an initial methionine amino acid residue.

The polynucleotides and polypeptides of the present invention may be employed as research reagents and materials for discovery of treatments and diagnostics for human disease.

In this same manner, HTTER36 (GDF3) and soluble fragments thereof can be employed as an anti-neoplastic compound, since members of this family show anti-proliferative effects on transformed cells. For in vivo use, the subject polypeptide may be administered in a variety of ways, including but not limited to, injection, infusion, topically, parenterally, etc. Administration may be in any physiologically acceptable carrier, including phosphate buffered saline, saline, sterilized water, etc.

A significant treatment involving HTTER36 (GDF3) and soluble fragments thereof relates to weight gain, particularly under high fat conditions. The polynucleotides, polypeptides, and compositions of the present invention may be employed for treating a variety of diseases and/or wasting conditions caused by severe treatment regimes. Some diseases that may be treated with HTTER36 (GDF3) include cachexia, AWS, GWS, anorexia nervosa, bulemia and other eating disorders, low fetal weight and low maternal weight, and other wasting conditions. The polynucleotides, polypeptides, and compositions of the invention may also be administered in conjunction with current disease treatments and therapies, as well as in a multidrug fashion to increase the effectiveness of HTTER36 (GDF3) and/or to induce complementary effects to lean muscle gain with another compound or an analog or derivative of HTTER36 (GDF3). HTTER (GDF3) and soluble fragments thereof may be incorporated in physiologically-acceptable carriers for patient administration. The nature of the carriers may vary widely.

The concentration of HTTER36 (GDF3) in the treatment composition is not critical but should be enough to induce appetite and/or weight gain.

The amount employed of the subject polypeptide will vary with the manner of administration, the employment of other active compounds, and the like, generally being in the range of about 1 μg to 100 μg. The subject polypeptide may be employed with a physiologically acceptable carrier, such as saline, phosphate-buffered saline, or the like. The amount of compound employed will be determined empirically, based on the response of cells in vitro and response of experimental animals to the subject polypeptides or formulations containing the subject polypeptides.

HTTER36 (GDF3) and soluble fragments thereof may be employed as a multi-functional regulator of cell growth and differentiation being capable of such diverse effects of inhibiting the growth of several human cancer cell lines, and T and B lymphocytes

HTTER36 (GDF3) and soluble fragments thereof may also be employed to inhibit early hematopoietic progenitor cell proliferation, stimulate the induction of differentiation of rat muscle mesenchymal cells, stimulate the differentiation, replication, and production of adipose tissue as well as the storage of energy-rich carbohydrates as fat, and stimulate production of cartilage-specific macro molecules, causing increased synthesis and secretion of collagen.

A limited sampling of HTTER36 (GDF3) mRNA levels in human adipose RNAs found a severely obese (BMI 37) sample having twice the normal GDF3 level (data not shown). Accordingly, patients with a predisposition of deregulated HTTER36 (GDF3) expression could develop obesity more readily. Thus, in a preferred aspect of the invention; HTTER36 (GDF3) may also be employed as a diagnostic tool where an overexpression, or deregulation, of HTTER36 (GDF3) would likely correlate with an increased likelihood of becoming obese.

This invention provides a method of screening compounds to identify antagonist compounds to the polypeptide of the present invention. As an example, a mammalian cell or membrane preparation expressing a HTTER36 (GDF3) receptor is incubated with a potential compound and the ability of the compound to generate a second signal from the receptor is measured to determine if it is an effective antagonist. Such second messenger systems include but are not limited to, cAMP guanylate cyclase, ion channels or phosphoinositide hydrolysis. Effective antagonists are also determined by the method above wherein an antagonist compound is detected which binds to the receptor but does not elicit a second messenger response to thereby block the receptor from HTTER36 (GDF3).

Another assay for identifying potential antagonists specific to the receptors to the polypeptide of the present invention is a competition assay, which comprises isolating plasma membranes that over-express a receptor to the polypeptide of the present invention, for example, human A431 carcinoma cells. Serially diluted test sample in a medium (volume is approximately 10 microliters) containing 10 nM ¹²⁵I-HTTER36 is added to five micrograms of the plasma membrane in the presence of the potential antagonist compound and incubated for 4 hours at 4° C. The reaction mixtures are diluted and immediately passed through a millipore filter. The filters are then rapidly washed and the bound radioactivity is measured in a gamma counter. The amount of bound HTTER36 is then measured. A control assay is also performed in the absence of the compound to determine if the antagonists reduce the amount of bound HTTER36.

Potential antagonist compounds include an antibody, or in some cases, an oligopeptide, which binds to the polypeptide. Alternatively, a potential antagonist may be a closely related protein that binds to the receptor, which is an inactive form of the polypeptide, and thereby prevent the action of the polypeptide of the present invention.

Another antagonist compound is an antisense construct prepared using antisense technology. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al, Science, 241:456 (1988); and Dervan et al., Science, 251: 1360 (1991)), thereby preventing transcription and the production of the polypeptide of the present invention. The antisense RNA oligonucleotide hybridizes to the mRNA in vivo and blocks translation of the mRNA molecule into the polypeptide of the present invention (Antisense-Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the antisense RNA or DNA may be expressed in vivo to inhibit production of the polypeptide of the present invention.

Antagonist compounds include a small molecule that binds to the polypeptide of the present invention and blocks its action at the receptor such that normal biological activity is prevented. The small molecules may also bind the receptor to the polypeptide to prevent binding. Examples of small molecules include but are not limited to small peptides or peptide-like molecules.

The antagonists may be employed to treat or prevent obesity. In other preferred embodiments, the antagonists of the invention may be employed to treat or prevent excessive appetite, metabolic syndrome (insulin resistance, alterations in glucose and lipid metabolism, increased blood pressure and visceral obesity), menopausal associated weight gain, excessive pregnancy weight gain, mental and psychological disorders such as bipolar disorder, depression, or schizophrenia, weight gain associated with the use of alterations in SNS effects on metabolism, high leptin levels in adolescent females, low perinatal birth weight (leading to childhood morbidity, such as diabetes), and changes in blood pressure such as increased blood pressure and increased incidence of hypertension.

The antagonists may also be employed to prevent the differentiation, replication, and production of adipose tissue as well as the storage of energy-rich carbohydrates as fat. Accordingly, the antagonists of the invention may be used to effect weight loss in a patient.

The antagonists of the invention may also be employed to prevent lipogenesis in liver. Thus, the antagonists of the invention may be used to prevent or treat steatosis of the liver associated with extreme obesity, uncontrolled diabetes/insulin resistance, hyperlipidemia, steroid use, acute starvation, rapid weight loss, intestinal bypass, and alcoholism.

The polypeptides of the present invention, agonist, or antagonist compounds may be employed in combination with a suitable pharmaceutical carrier. Such compositions comprise a therapeutically effective amount of the polypeptide or compound, and a pharmaceutically acceptable carrier or excipient. Such a carrier includes but is not limited to saline, buffered saline, dextrose, water, glycerol, ethanol, and combinations thereof. The formulation should suit the mode of administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides or compounds of the present invention may be employed in conjunction with other therapeutic compounds.

The pharmaceutical compositions may be administered in a convenient manner such as by the oral, topical, intravenous, intraperitoneal, intramuscular, subcutaneous, intranasal or intradermal routes. The pharmaceutical compositions are administered in an amount that is effective for treating and/or prophylaxis of the specific indication. In general, they are administered in an amount of at least about 10 μg/kg body weight and in most cases they will be administered in an amount not in excess of about 8 mg/Kg body weight per day. In most cases, the dosage is from about 10 μg/kg to about 1 mg/kg body weight daily, taking into account the routes of administration, symptoms, etc.

The polypeptides, and antagonists which are polypeptides, may also be employed in accordance with the present invention by expression of such polypeptides in vivo, which is often referred to as “gene therapy.”

Thus, for example, cells from a patient may be engineered with a polynucleotide (DNA or RNA) encoding a polypeptide ex vivo, with the engineered cells then being provided to a patient to be treated with the polypeptide. Such methods are well-known in the art and are apparent from the teachings herein. For example, cells may be engineered by the use of a retroviral plasmid vector containing RNA encoding a polypeptide of the present invention.

Similarly, cells may be engineered in vivo for expression of a polypeptide in vivo by, for example, procedures known in the art. For example, a packaging cell is transduced with a retroviral plasmid vector containing RNA encoding a polypeptide of the present invention such that the packaging cell now produces infectious viral particles containing the gene of interest. These producer cells may be administered to a patient for engineering cells in vivo and expression of the polypeptide in vivo. These and other methods for administering a polypeptide of the present invention by such method should be apparent to those skilled in the art from the teachings of the present invention.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, No. 9, 980-990 (1989), or any other promoter (e.g., cellular promoters such as eukaryotic cellular promoters including, but not limited to, the histone, pol III, and β-actin promoters). Other viral promoters that may be employed include, but are not limited to, adenovirus promoters, thymidine kinase (TIC) promoters, and B19 parvovirus promoters. The selection of a suitable promoter will be apparent to those skilled in the art from the teachings contained herein.

The nucleic acid sequence encoding the polypeptide of the present invention is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter, heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters. The promoter also may be the native promoter that controls the gene encoding the polypeptide.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles that include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells that may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

This invention is also related to the use of the gene of the present invention as a diagnostic. Detection of a mutated form of the gene of the present invention will allow a diagnosis of a disease or a susceptibility to a disease which results from under expression of the polypeptide of the present invention, for example, low maternal weight and low fetal weight as well as indication and/or confirmation of an eating disorder.

Individuals carrying mutations in the human gene of the present invention may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR (Saiki et al., Nature, 324:163-166 (1986)) prior to analysis. RNA or cDNA may also be used for the same purpose. As an example, PCR primers complementary to the nucleic acid encoding a polypeptide of the present invention can be used to identify and analyze mutations thereof. For example, deletions and insertions can be detected by a change in size of the amplified product in comparison to the normal genotype. Point mutations can be identified by hybridizing amplified DNA to radiolabeled RNA or alternatively, radiolabeled antisense DNA sequences. Perfectly matched sequences can be distinguished from mismatched duplexes by RNase A digestion or by differences in melting temperatures.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobility of different DNA fragments are retarded in the gel at different positions according to their specific melting or partial melting temperatures (see, e.g., Myers et al., Science, 230:1242 (1985)).

Sequence changes at specific locations may also be revealed by nuclease protection assays, such as RNase and S1 protection or the chemical cleavage method (e.g., Cotton et al., PNAS, USA, 85:4397-4401 (1985)).

Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

The present invention also relates to diagnostic assays for detecting altered levels of the polypeptide of the present invention in various tissues since an over-expression of the proteins compared to normal control tissue samples can detect the presence of certain disease conditions such as a propensity towards obesity. Conversely, underexpression of the proteins of the invention compared to normal control tissue samples can detect the presence of certain disease conditions such as low maternal weight and low fetal weight as well as indication and/or confirmation of an eating disorder.

Assays used to detect levels of the polypeptide of the present invention in a sample derived from a host are well-known to those of skill in the art and include radioimmunoassays, competitive-binding assays, Western Blot analysis and preferably an ELISA assay. An ELISA assay initially comprises preparing an antibody specific to an antigen of the polypeptide of the present invention, preferably a monoclonal antibody. In addition a reporter antibody is prepared against the monoclonal antibody. To the reporter antibody is attached a detectable reagent such as radioactivity, fluorescence or in this example a horseradish peroxidase enzyme. A sample is now removed from a host and incubated on a solid support, e.g. a polystyrene dish, which binds the proteins in the sample. Any free protein binding sites on the dish are then covered by incubating with a non-specific protein such as bovine serum albumin. Next, the monoclonal antibody is incubated in the dish during which time the monoclonal antibodies attach to any polypeptides of the present invention attached to the polystyrene dish. All unbound monoclonal antibody is washed out with buffer. The reporter antibody linked to horseradish peroxidase is now placed in the dish resulting in binding of the reporter antibody to any monoclonal antibody bound to polypeptides of the present invention. Unattached reporter antibody is then washed out. Peroxidase substrates are then added to the dish and the amount of color developed in a given time period is a measurement of the amount of protein present in a given volume of patient sample when compared against a standard curve.

A competition assay may also be employed to determine levels of the polypeptide of the present invention in a sample derived from the hosts. Such an assay comprises isolating plasma membranes that over-express the receptor for the polypeptide of the present invention. A test sample containing the polypeptides of the present invention that have been labeled, are then added to the plasma membranes and then incubated for a set period of time. Also added to the reaction mixture is a sample derived from a host that is suspected of containing the polypeptide of the present invention. The reaction mixtures are then passed through a filter that is rapidly washed and the bound radioactivity is then measured to determine the amount of competition for the receptors and therefore the amount of the polypeptides of the present invention in the sample.

Antibodies specific to HTTER36 (GDF3) may be used for cancer diagnosis and therapy, since many types of cancer cells up-regulate various members of this super family during the process of neoplasia or hyperplasia. These antibodies bind to and inactivate HTTER36 (GDF3). Monoclonal antibodies against HTTER36 (GDF3) (and/or its family members) are in clinical use for both the diagnosis and therapy of certain disorders including (but not limited to) hyperplastic and neoplastic growth abnormalities. Up-regulation of growth factor expression by neoplastic tissues forms the basis for a variety of serum assays that detect increases in growth factor in the blood of affected patients. These assays are typically applied not only in diagnostic settings, but are applied in prognostic settings as well (to detect the presence of occult tumor cells following surgery, chemotherapy, etc).

In addition, malignant cells expressing the HTTER36 (GDF3) receptor may be detected by using labeled HTTER36 (GDF3) in a receptor binding assay, or by the use of antibodies to the HTTER36 (GDF3) receptor itself. Cells may be distinguished in accordance with the presence and density of receptors for HTTER36 (GDF3), thereby providing a means for predicting the susceptibility of such cells to the biological activities of HTTER36 (GDF3).

Antibodies specific to HTTER36 (GDF3) may also be used for diagnosis of obesity and the treatment thereof, since elevated levels of HTTER36 (GDF) mRNA are found in severely obese samples (data not shown). These antibodies bind to and inactivate HTTER36 (GDF3). Thus, monoclonal antibodies against HTTER36 (GDF3) may also be particularly useful in the diagnosis and treatment of obesity and obesity-related disorders.

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique that provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic mice may be used to express humanized antibodies to immunogenic polypeptide products of this invention.

The present invention will be further described with reference to the following examples; however, it is to be understood that the present invention is not limited to such examples. All parts or amounts, unless otherwise specified, are by weight.

In order to facilitate understanding of the following examples certain frequently occurring methods and/or terms will be described.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37° C. are ordinarily used, but may vary in accordance with the supplier's instructions. After digestion the reaction is electrophoresed directly on a polyacrylamide gel to isolate the desired fragment.

Size separation of the cleaved fragments is performed using 8 percent polyacrylamide gel described by Goeddel, D. et al., Nucleic Acids Res., 8:4057 (1980).

“Oligonucleotides” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis, T., at al., Id., p. 146). Unless otherwise provided, ligation may be accomplished using known buffers and conditions with 10 units of T4 DNA ligase (“ligase”) per 0.5 μg of approximately equimolar amounts of the DNA fragments to be ligated.

Unless otherwise stated, transformation was performed as described in the method of Graham, F. and Van der Eb, A., Virology, 52:456-457 (1973).

### Example 1

### Bacterial Expression and Purification of mature HTTER36 (GDF3)

The DNA sequence encoding HTTER36 (GDF3), ATCC # 97349, was initially amplified using PCR oligonucleotide primers corresponding to the 5′ sequences of the processed HTTER36 protein and the vector sequences 3′ to the HTTER36 gene. Additional nucleotides corresponding to HTTER36 were added to the 5′ and 3′ sequences respectively. The 5′ oligonucleotide primer has the sequence 5′ GAAAGGATCCGCAGCCATCCCTGTCCCCAAACTTTCTTGT 3′ (SEQ ID NO:3) contains a BamHI restriction enzyme site (in bold) followed by 18 nucleotides of HTTER36 coding sequence starting from nucleotide 791 of FIGS. 1A-B (SEQ ID NO:1). The 3′ sequence 5′ TCCTTCTATTCAAGCTTCTGACATCCTACCCACACCCACA 3′ (SEQ ID NO:4) contains complementary sequences to a Hind III site and is followed by 15 nucleotides of HTTER36 beginning at nucleotide 1121, and a stop codon. The restriction enzyme sites correspond to the restriction enzyme sites on the bacterial expression vector pQE-9 (Qiagen, Inc. Chatsworth, Calif., 91311). pQE-9 encodes antibiotic resistance (Amp'), a bacterial origin of replication (ori), an IPTG-regulatable promoter operator (IVO), a ribosome binding site (RBS), a 6-His tag and restriction enzyme sites. pQE-9 was then digested with BamHI and Hind III.

The amplified sequences were ligated into pQE-9 and were inserted in frame with the sequence encoding for the histidine tag and the RBS. The ligation mixture was then used to transform E. coli strain DH5 alpha (Gibco BRL) the procedure described in Sambrook, J. et al., Molecular Cloning: A Laboratory Manual, Cold Spring Laboratory Press, (1989). Transformants were identified by their ability to grow on LB plates and ampicillin/kanamycin resistant colonies were selected. Plasmid DNA was isolated and confirmed by restriction analysis. Clones containing the desired constructs were grown overnight (O/N) in liquid culture in LB media supplemented with both Amp (100 ug/ml) and Kan (25 ug/ml). The O/N culture was used to inoculate a large culture at a ratio of 1:100 to 1:250. The cells were grown to an optical density 600 (O.D.⁶⁰⁰) of between 0.4 and 0.6. IPTG (“Isopropyl-B-D-thiogalacto pyranoside”) was then added to a final concentration of 1 mM. IPTG induces by inactivating the lad repressor, clearing the P/O leading to increased gene expression. Cells were grown an extra 3 to 4 hours. Cells were then harvested by centrifugation. The cell pellet was solubilized in the chaotropic agent 6 Molar Guanidine HCl.

After clarification, solubilized HTTER36 was purified from this solution by chromatography on a Nickel-Chelate column under conditions that allow for tight binding by proteins containing the 6-His tag (Hochuli, E. et al., S. Chromatography 411:177-184 (1984)). HTTER36 (85% pure) was eluted from the column in 6 molar guanidine HCl pH 5.0 and for the purpose of renaturation adjusted to 3 molar guanidine HCl, 100 mM sodium phosphate, 10 molar glutathione (reduced) and 2 molar glutathione (oxidized). After incubation in this solution for 12 hours the protein was dialyzed to 10 molar sodium phosphate.

### Example 2

### Cloning and expression HTTER36 (GDF3) using the Baculovirus Expression System

The DNA sequence encoding the HTTER36 protein, ATCC # 97349, is amplified using PCR oligonucleotide primers corresponding to the 5′ and 3′ sequences of the gene.

The primers used are: 5′ CAGGGATCCGCCATCATGCTTCGTTTCTTGCCAGA 3′ (SEQ ID NO:5) contains the underlined Bam HI site an efficient signal for the initiation of translation in eukaryotic cells, a start codon (bold) and 17 bps of HTTER36 (GDF3) coding sequence. The 3′ primer has the sequence 5′ CTTCGGTACCCATTTCTGACATCCTACCCACAC 3′ (SEQ ID NO:6) contains the underlined Asp718 site, and 23 nucleotides complementary to the 3′ end of the HTTER36 (GDF3) sequence beginning at nucleotide 1126.

The amplified sequences are isolated from a 1% agarose gel using a commercially available kit (“Geneclean,” BIO 101 Inc., La Jolla, Calif.). The fragment is then digested with the endonucleases BamHI and Asp718 and then purified again on a 1% agarose gel. This fragment is designated F2.

The vector pA2 is used (modification of pVL941 vector, discussed below) for the expression of the HTTER36 (GDF3) protein using the baculovirus expression system (for review see: Summers, M. D. and Smith, G. E. 1987, A manual of methods for baculovirus vectors and insect cell culture procedures, Texas Agricultural Experimental Station Bulletin No. 1555). This expression vector contains the strong polyhedrin promoter of the Autographa californica nuclear polyhedrosis virus (AcMNPV) followed by the recognition sites for the restriction endonucleases. The polyadenylation site of the simian virus (SV)40 is used for efficient polyadenylation. For an easy selection of recombinant virus the beta-galactosidase gene from E. coli is inserted in the same orientation as the polyhedrin promoter followed by the polyadenylation signal of the polyhedrin gene. The polyhedrin sequences are flanked at both sides by viral sequences for the cell-mediated homologous recombination of co-transfected wild-type viral DNA. Many other baculovirus vectors could be used such as pAc373, pRG1, pVL941 and pAcIM1 (Luckow, V. A. and Summers, M. D., Virology, 170:31-39).

The plasmid is digested with the restriction enzymes BamHI and Asp718 and then dephosphorylated using calf intestinal phosphatase by procedures known in the art. The DNA is then isolated from a 1% agarose gel using the commercially available kit (“Geneclean” BIO 101 Inc., La Jolla, Calif.). This vector DNA is designated V2.

Fragment F2 and the dephosphorylated plasmid V2 are ligated with T4 DNA ligase. E. coli HB101 cells are then transformed and bacteria identified that contained the plasmid (pBacHTTER36) with the HTTER36 (GDF3) gene using the restriction enzymes BamHI and Asp718. The sequence of the cloned fragment is confirmed by DNA sequencing.

5 μg of the plasmid pBacHTTER36 is co-transfected with 1.0 μg of a commercially available linearized baculovirus (“BaculoGold™ baculovirus DNA”, Pharmingen, San Diego, Calif.) using the lipofection method (Feigner et al. Proc. Natl. Acad. Sci. USA, 84:7413-7417 (1987)).

1 μg of BaculoGold™ virus DNA and 5 μg of the plasmid pBacHTTER36 are mixed in a sterile well of a microliter plate containing 50 μl of serum free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added drop-wise to the Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation continued at 27° C. for four days.

After four days the supernatant is collected and a plaque assay performed similar as described by Summers and Smith (supra). As a modification an agarose gel with “Blue Gal” (Life Technologies Inc., Gaithersburg) is used which allows an easy isolation of blue stained plaques. (A detailed description of a “plaque assay” can also be found in the user's guide for insect cell culture and baculovirology distributed by Life Technologies Inc., Gaithersburg, page 9-10).

Four days after the serial dilution, the virus is added to the cells and blue stained plaques are picked with the tip of an Eppendorf pipette. The agar containing the recombinant viruses is then resuspended in an Eppendorf tube containing 200 μl of Grace's medium. The agar is removed by a brief centrifugation and the supernatant containing the recombinant baculovirus is used to infect Sf9 cells seeded in 35 mm dishes. Four days later the supernatants of these culture dishes are harvested and then stored at 4° C.

Sf9 cells are grown in Grace's medium supplemented with 10% heat-inactivated FBS. The cells are infected with the recombinant baculovirus V-HTTER36 at a multiplicity of infection (MOI) of 2. Six hours later the medium is removed and replaced with SF900 II medium minus methionine and cysteine (Life Technologies Inc., Gaithersburg). 42 hours later 5 μCi of ³⁵S-methionine and 5 μCi ³⁵S cysteine (Amersham) are added. The cells are further incubated for 16 hours before they are harvested by centrifugation and the labeled proteins visualized by SDS-PAGE and autoradiography.

### Example 3

### Expression of Recombinant HTTER36 (GDF3) in CHO Cells

The vector pC1 is used for the expression of the HTTER36 (GDF3) protein. Plasmid pC1 is a derivative of the plasmid pSV2-dhfr [ATCC Accession No. 37146]. Both plasmids contain the mouse dhfr gene under control of the SV40 early promoter. Chinese hamster ovary- or other cells lacking dihydrofolate activity that are transfected with these plasmids can be selected by growing the cells in a selective medium (alpha minus MEM, Lift Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology Vol. 9:64-68). Cells grown in increasing concentrations of MTX develop resistance to the drug by overproducing the target enzyme, DHFR, as a result of amplification of the DHFR gene. If a second gene is linked to the dhfr gene it is usually co-amplified and over-expressed. It is state of the art to develop cell lines carrying more than 1,000 copies of the genes. Subsequently, when the methotrexate is withdrawn, cell lines contain the amplified gene integrated into the chromosome(s).

Plasmid pN346 contains for the expression of the gene of interest a strong promoter of the long terminal repeat (LTR) of the Rouse Sarcoma Virus (Cullen, et al., Molecular and Cellular Biology, March 1985, 438-447) plus a fragment isolated from the enhancer of the immediate early gene of human cytomegalovirus (CMV) (Boshart et al., Cell 41:521-530, 1985). Downstream of the promoter are the following single restriction enzyme cleavage sites that allow the integration of the genes: BamHI, PvuII, and NruI. Behind these cloning sites the plasmid contains translational stop codons in all three reading frames followed by the 3′ intron and the polyadenylation site of the rat preproinsulin gene. Other high efficient promoters can also be used for the expression, e.g., the human β-actin promoter, the SV40 early or late promoters or the long terminal repeats from other retroviruses, e.g., HIV and HTLVI. For the polyadenylation of the mRNA other signals, e.g., from the human growth hormone or globin genes can be used as well.

Stable cell lines carrying a gene of interest integrated into the chromosome can also be selected upon co-transfection with a selectable marker such as gpt, G418 or hygromycin. It is advantageous to use more than one selectable marker in the beginning, e.g. G418 plus methotrexate.

The plasmid pN346 was digested with the restriction enzyme BamHI and then dephosphorylated using calf intestinal phosphatase by procedures known in the art. The vector was then isolated from a 1% agarose gel.

The DNA sequence encoding HTTER36 (GDF3), ATCC # 97349 was amplified using PCR oligonucleotide primers corresponding to the 5′ and 3′ sequences of the gene:

The 5′ primer has the sequence 5′ ACAGCGGATCCAGCCACC ATGCTTCGTTTCTTGCCA 3′ (SEQ ID NO:7) and contains a BamHI restriction enzyme site (in bold) followed by an efficient signal for translation (Kozak, M., supra) plus the first 18 nucleotides of the gene (the initiation codon for translation “ATG” is underlined).

The 3′ primer has the sequence 5′ TCCTTCGGATCCCATTTCT GACATCCTACCCACACCCACA 3′ (SEQ ID NO:8) and contains the cleavage site for the restriction endonuclease Bam HI and 29 nucleotides complementary to the 3′ translated and non-translated sequence of the gene.

The amplified fragments were isolated from a 1% agarose gel as described above and then digested with the endonuclease BglII and then purified again on a 1% agarose gel.

The isolated fragment and the dephosphorylated vector were then ligated with T4 DNA ligase. E. coli HB101 cells were then transformed and bacteria identified that contained the plasmid pN346 inserted in the correct orientation using the restriction enzyme BamHI. The sequence of the inserted gene was confirmed by DNA sequencing.

### Transfection of CHO-dhfr-cells

Chinese hamster ovary cells lacking an active DHFR enzyme were used for transfection. 5 μg of the expression plasmid N346 were cotransfected with 0.5 μg of the plasmid pSVneo using the lipofectin method (Feigner et al., supra). The plasmid pSV2-neo contains a dominant selectable marker, the gene neo from Tn5 encoding an enzyme that confers resistance to a group of antibiotics including G418. The cells were seeded in alpha minus MEM supplemented with 1 mg/ml G418. After 2 days, the cells were trypsinized and seeded in hybridoma cloning plates (Greiner, Germany) and cultivated from 10-14 days. After this period, single clones were trypsinized and then seeded in 6-well petri dishes using different concentrations of methotrexate (25, 50 nm, 100 nm, 200 nm, 400 nm). Clones growing at the highest concentrations of methotrexate were then transferred to new 6-well plates containing even higher concentrations of methotrexate (500 nM, 1 μM, 2 μM, 5 μM). The same procedure was repeated until clones grew at a concentration of 100 μM.

The expression of the desired gene product was analyzed by Western blot analysis and SDS-PAGE.

### Example 4

### Expression Via Gene Therapy

Fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin, is added. This is then incubated at 37° C. for approximately one week. At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al, DNA, 7:219-25 (1988) flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention is amplified using PCR primers that correspond to the 5′ and 3′ end sequences respectively. The 5′ primer containing an EcoRI site and the 3′ primer further includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is used to transform bacteria HB101, which are then plated onto agar-containing kanamycin for the purpose of confirming that the vector had the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells are transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his.

The engineered fibroblasts are then injected into the host, either alone or after having been grown to confluence on cytodex 3 microcarrier beads. The fibroblasts now produce the protein product.

### Example 5

### Effects of HTTER36 (GDF3) on Th1/Th2 Differentiation

To determine the effect of HTTER36 (GDF3) on human Th1/Th2 differentiation an assay where naive human DCD4+ T cells are induced to differentiate under neutral (Th0), Th1 or Th2 conditions was used. Naive CD4, CD45RAT cells are purified from human cord blood (Poietic Technologies, Germantown, Md.) and cultured (0.75×10 6 cells/750 μl) in 24 well plates in RPMI-1640-10% FCS in the presence of the T cell mitogen PHA (1 ug/ml) under the following conditions:

Neutral: medium containing isotype matched control mAB (murine IgG1 from Cappell)

Th1 directed: in the presence of IL-12 (0.1 ng/ml) and anti-M-4 (mAB 5A4 ascites 1:200)

Th2 directed: in the presence of IL-4 (0.1 ng/ml) and anti-IL-12 (mAb C.8.6, 1 ug/ml).

HTTER36 (GDF3) and positive controls (IL-12, 5 ng/ml for Th1 and IL-4, 5 ng/ml for Th2) are added at the initiation of culture. After 5 days of culture at 37 C the plates are spun down and the supernatants removed. The cells are then restimulated with fresh medium containing stimulatory anti-CD3 (HIT3a 1 μg/ml) and IL-2 (10 U/ml) HTTER36 (GDF3) or positive/negative controls, but omitting the directing cytokines and antibodies. After an additional 48 hours of culture at 37° C. the plates are spun down and supernatants measured for IFN-γ (Th1) and IL-4 (Th2) by ELISA.

In this experiment, the positive control (IL-12) induced IFNγ production under neutral, Th1 conditions and Th2 conditions. In this experiment culture medium alone, under Th1 directed conditions also resulted in significant IFNγ production. IL-4 also induced high levels of IFNγ under Th1 conditions. HTTER36 (GDF3) also induced IFNγ production above that observed with culture medium alone, but only under Th1 directed conditions with an optimal response at 1 ng/ml. This effect cannot be attributed to endotoxin, a potent inducer of IL-12, because it was not observed under Th0 conditions. No effect on IL-4 production has been observed with HTTER36 (GDF3).

### Example 6

### Adenoviral Expression of HTTER36

A. HTTER36 (GDF3) Adenoviruses

Human HTTER36 (GDF3) open reading frame was amplified by PCR with two primers with the sequences 5′-CGGTGCTCTAGACCGCCATCATGCTTCGTTTCTTGCCAGATTTGGC-3′ and 5′-GTCGTCGGTACCTTACCCACACCCACATTCATCGACTAC-3′ using a full length GDF-3 cDNA clone (HTTER36) isolated from a teratocarcinoma cDNA library as the template. The PCR product was digested with restriction enzymes XbaI and Asp718 and ligated to pShuttle2 vector in the Adeno-X Expression System kit (BD Clontech, Ca.) to generate a shuttle vector pShuttle2:GDF3. The HTTER36 (GDF3) expression cassette in pShuttle2:GDF3 was excised by I-CeuI and PI-Scel restriction digestion and grafted into. Adeno-X viral DNA predigested with PI-Sce I/I-Ceu to produce adx:GDF3, the recombinant adenoviral DNA for GDF-3 expression. adx:GDF3 viruses were produced in HEK293A host cells and purified by BD Adeno-X Virus Purification Kit as per the manufacturer's instruction. The adx:GDF3 virus preparation had a titer of 2×10¹⁰ pfu/mL. Control adx:LacZ viruses which express β-galactosidase gene were amplified and purified from Adeno-X-LacZ Adenovirus (BD Clontech).

B. Verification of HTTER36 (GDF3) Gene Expression.

GDF3 gene expression was verified by HEK293A cells transduced with adx:GDF3. 1×10⁵HEK293 cells were infected with adx:GDF3 viruses at a MOI of 100 for one hour. The cells were refed with fresh DMEM medium supplemented with 10% fetal bovine serum and allowed for gene expression for five days. The cells were lysed in 0.2 mL SDS-PAGE sample buffer plus 0.1 mM PMSF, heated to 100° C. for five minutes and clarified by microcentrifugation. 10 μL lysate was resolved on SDS-polyacrylamide gel and immunoblotted with a rabbit anti-hGDF3 antibody developed with bacterially expressed polyhistidine-tagged full length human GDF3 protein as the antigen.

### Example 7

### In Vivo Expression of Adenoviral HTTER36 (GDF3) in Mice

A. In vivo Adenoviral HTTER36(GDF3) Delivery and Expression in Mice

Three-month-old wild type C57BL/6J male mice (Taconic, N.Y.) weighing approximately 20 grams were used in the study. Adx:GDF3 virions were reconstituted in 0.1 c.c. PBS and injected intravenously via tail vein at a dose of 1×10⁹ pfu per mouse. Adx:LacZ viruses at the same dose were used as a negative control. All animal studies were performed using approved protocols at Human Genome Sciences, Inc.

HTTER36 (GDF3) gene expression in adx:GDF3 transduced mice was determined by Taqman analysis of 25 ng liver total RNAs using Trizol RNA extraction method (Invitrogen, Ca.). Human HTTER36 (GDF3) Taqman primer pair specific for the adx:GDF3 transgene has the probe sequence of 5′. CTCCCAGACCAAGGTTTCTTTCTTFACCCAAA-3′ and primer sequences of 5′-CGTCCGCGGGAATGTACTT-3′ and 5′-CAGGAGGAAGCTTGGGAAATT-3′. Mouse glyceraldehyde 3-phosphate dehydrogenase (GAPDH) was used as an internal reference, with the probe sequence of 5′-CACTCACGGCAAATTCAACGGCAC-3′ and primer sequences of 5′-TACATGGTCTACATGTTCCAGTATGACT-3′ and 5′-TCCCATTCTCGGCCTTGAC-3′. Adx:GDF3 gene delivery produced sustained but not permanent HTTER36 (GDF3) expression for two months, with a peak expression ratio of 3.08E-2 over GAPDH.

B. Animal Body Weights, Histology and Adipocyte Hypertrophy

Methods:

Three-month-old, wild-type C57BL/6J male mice, matched for body weight, were randomly housed into diet-based experiment groups. Each group received either HTTER36 (GDF3) adenovirus (adx:GDF3) or negative control adenovirus (adx:LacZ) at 1×10⁹ pfu/mouse. The high fat diet groups were maintained in 60 kcal % high-fat diet (D12492, Research Diets Inc., NJ) and the normal chow groups in were maintained in a matching normal (10 kcal %) fat diet (D12450B) ad libitum with free access to water. Growth curves were recorded by weighing mice between 10:00 and 12:00 a.m.

Using standard histological procedures, tissues from white fat tissues and major organs were collected from each group, fixed in 10% neutral buffered formalin, embedded in paraffin and cut into 10 um thickness sections for histological analysis. The tissue sections were stained by hematoxylin/eosin, visualized and photographed under a microscope.

White fat tissue histology sections were microphotographed under a same magnification to determine adipocyte hypertrophy. Adipocyte hypertrophies were compared based on cell volume or cell mass. Adipocyte relative hypertrophy by cell volume (rHᵥ) is defined as the ratio of adipocyte cell volumes typically using normal adipocytes as the denominator. Cell numbers in randomly selected fields of one arbitrary unit area were counted and averaged as N. rHᵥ is approximately

$${( \sqrt{\frac{{N}_{0}}{N}} )}^{3} ,$$

where N₀ is the average of normal cell numbers.

Adipocyte relative hypertrophy by cell mass (rHₘ) is defined as the ratio of average adipocyte weights. Genomic DNA was extracted from 40 mg of white fat tissues using the DNeasy Tissue Kit (Qiagen) and concentration was determined by U.V. absorbance at OD₂₆₀. rHₘ is calculated as

$$\frac{M / DNA}{{M}_{0} / {DNA}_{0}} ,$$

where M is the tissue weight, DNA is the DNA content extracted from the tissue, and M₀ and DNA₀ are the values of normal adipose tissue.

Results and Discussion:

Body Weight Gain Induced by HTTER36 (GDF3) Overexpression. Mice in the experimental groups were given the following designations: GDF3/Fat=adx:GDF3 gene transfer and continuous 60% high fat diet; LacZ/Fat adx:LacZ gene transfer and continuous 60% high fat diet; GDF3/Chow=adx:GDF3 gene transfer and continuous normal chow; and LacZ/Chow=adx:LacZ gene transfer and continuous normal chow.

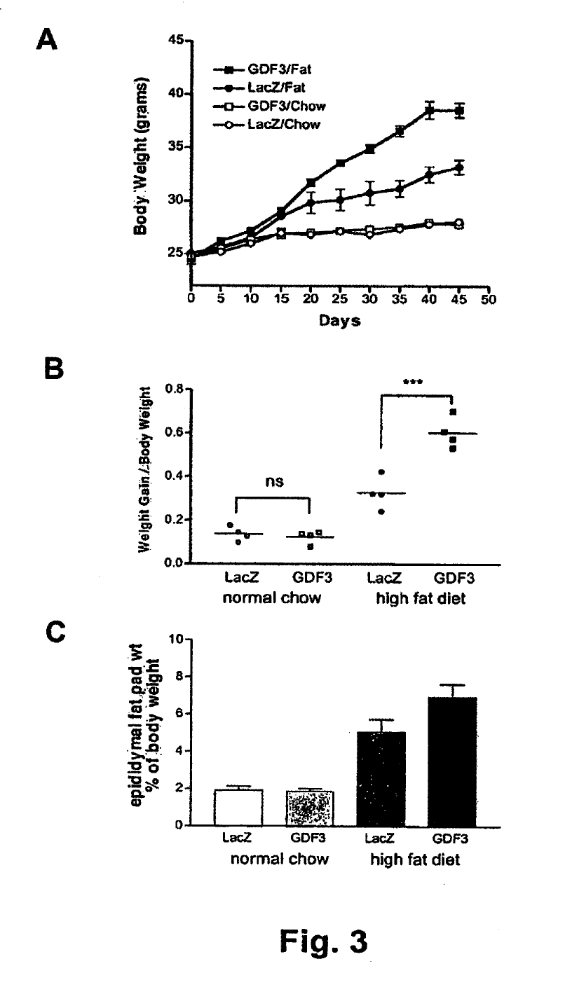

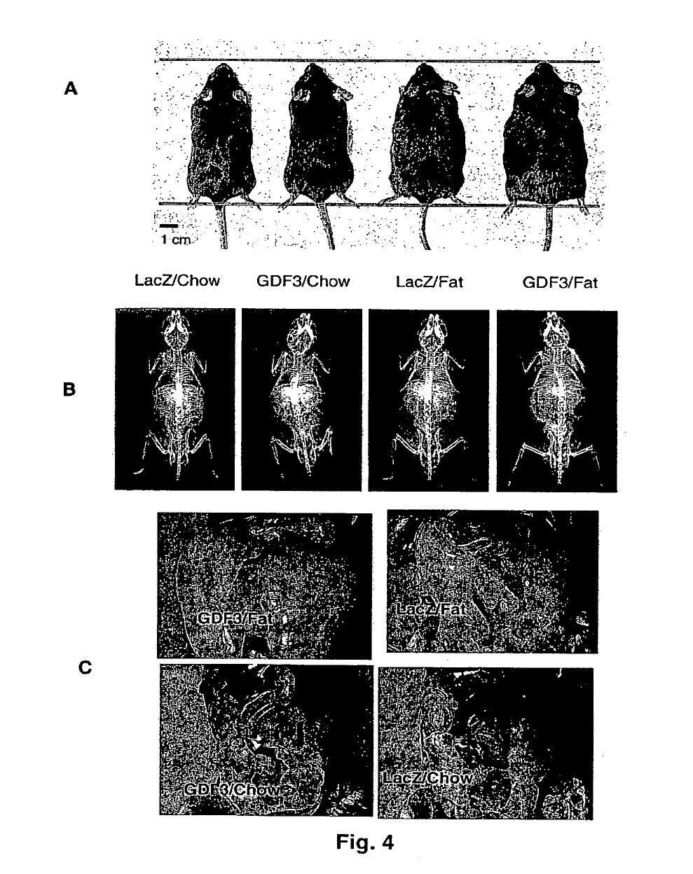

One-way ANOVA analysis showed no significant difference in initial body weights among the groups. The growth curves of each group are shown in FIG. 3A. High fat diet groups (GDF3/Fat and LacZ/Fat) had accelerated weight gains than normal chow groups (GDF3/Chow and LacZ/Chow). However, the GDF3/Fat mice outpaced the LacZ/Fat group to a greater extent (38±0.65 vs. 33±0.68 grams on day 45). GDF3/Fat mice had significantly more body weight gain than LacZ/Fat (P<0.001), and more so than any other groups (FIG. 3B). The GDF3/Fat mice were visibly more obese (FIG. 4A) and had profound increase of abdominal fat depots (FIG. 4C). No difference in weight gain between GDF3/Chow and LacZ/Chow was detected. Thus, the data indicates that HTTER36 (GDF3) promotes body weight gain and does so only under high fat dietary condition.

Gross anatomy did not reveal obvious changes in shape and size of heart, lung, kidney, spleen, digestive track, liver, pancreas, or muscle among the experiment groups. Total body X-ray imaging showed no craniofacial, axial, extremity and other skeletal abnormalities. The head and body lengths as well as overall skeletal frames were also not dissimilar. Thus, this data suggests that GDF-3, as a bone morphogenetic protein family member, is not involved in skeletal function (FIG. 4B). Together with the greater adiposity predicted by epididymal fat pad weights (FIG. 3C), this data also suggests that the increased weight gain in GDF3/Fat mice is attributed mainly to adipose expansion.

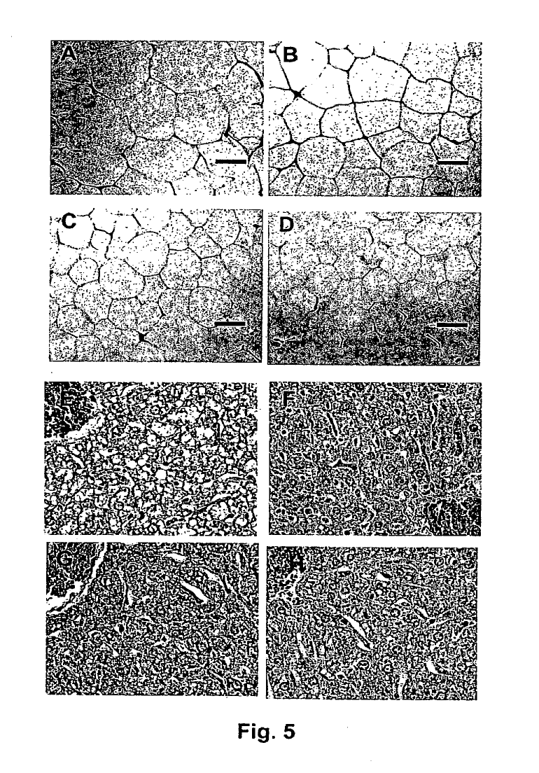

Adipocyte Hypertrophy Induced by HTTER36 (GDF3) Overexpression. Histological examination of tissue samples from each group showed prominent adipocyte hypertrophy in GDF3/Fat mice. Adipocyte hypertrophy was less in LacZ/Fat mice and lacking in GDF3/Chow and LacZ/Chow mice (FIG. 5A, B, C, D).

The degrees of adipocyte hypertrophies were compared in terms of both cell volume size (rHᵥ) and cell mass (rHₘ) using the LacZ/Chow adipocyte as the reference (Table 1). The highly hypertrophic GDF3/Fat adipocytes were laden with fat deposit that could be the result of increased lipid synthesis, lipid influx and/or reduced lipolysis or lipid efflux. With the exception of liver, other tissues including skeletal muscle, kidney and bone had no obvious abnormalities in each experiment group. The increased body weight gain, the expansion of white fat tissue and the adipocyte hypertrophy in GDF3/Fat mice are in agreement with an adipogenic function by HTTER36 (GDF3). The necessity of high fat diet for HTTER36 (GDF3) to exhibit adipogenic effect reveals an underlying relationship between fat metabolism and adipose regulation by HTTER36 (GDF3). This data further supports an interplay between GDF-3 and fat metabolism suggested by B. A. Witthuln and D. A. Bernlohr (Cytokine, 14:129-135 (2001)), who showed that a high fat diet stimulates HTTER36 (GDF3) expression in aP2 null mice but abolishes it in wild type mice.

TABLE 1

| Hypertrophy | LacZ/Chow | GDF3/Chow | LacZ/Fat | GDF3/Fat |
|---|---|---|---|---|
| by volume | 1 | 1.08 ± 0.05 | 2.15 ± 0.13 | 5.30 ± 0.27 |
| (rHᵥ) | | | | |
| by mass | 1 | 0.93 ± 0.06 | 1.54 ± 0.09 | 1.94 ± 0.13 |
| (rHₘ) | | | | |

Relative hypertrophies of LacZ/Chow, GDF3/Chow, LacZ/Fat, and GDF3/Fat adipocytes.
Values are expressed as the mean ± SEM (n = 8 for rHᵥ, n = 4 for rHₘ).

Hepatic Steatosis. In addition to the effects on adipose tissue, GDF3/Fat liver underwent marked steatosis development (FIG. 5E). FIG. 5E shows that hepatocytes packed with fat vacuoles were localized in all three zones of the liver lobules. The trabecular pattern of the liver lobules was blurred in the affected area. While steatosis was also very mildly induced by high fat diet alone (LacZ/Fat group), the hepatocytes distended by fat were sporadically dispersed in zone I, far less in number, much smaller in fat vacuole size, and did not disrupt liver lobule trabecular structure (FIG. 5F). Normal chow groups (GDF3/Chow and LacZ/Chow) had entirely normal liver histology (FIGS. 5G and H). There were no apparent lipid infiltration or structural disruption by fat in other tissues such as skeletal muscle, bone, kidney, and spleen in all groups.

C. Serum Leptin Levels

Methods:

Serum leptin were determined by Quantikine Mouse Leptin Immunoassay kit (R&D systems, MN) according to the manufacturers' instructions. All measurements were done in triplicates. Raw assay values were converted to leptin or insulin concentrations by standard reference curves and sample dilution factors.

Results and Discussion:

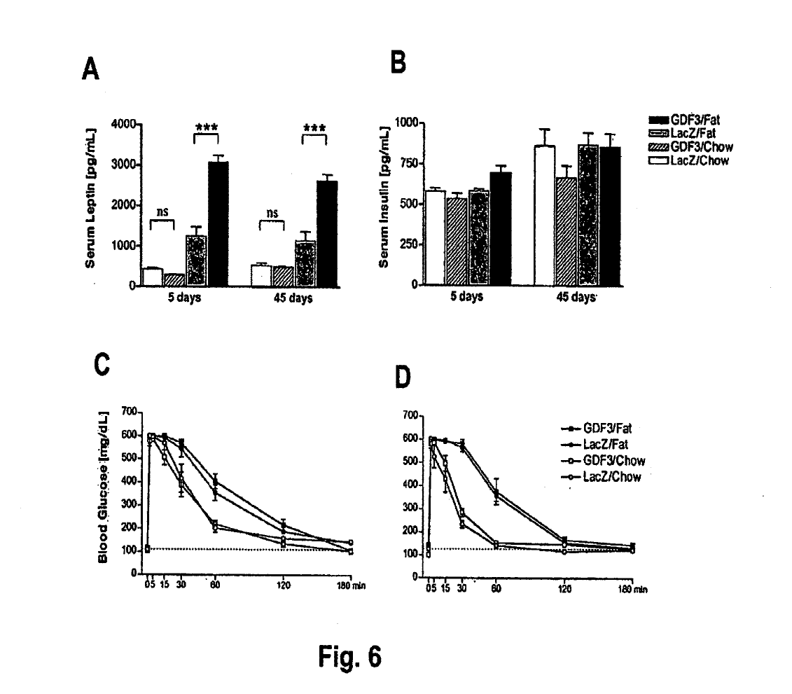

HTTER36 (GDF3) by itself did not increase serum leptin in normal diet groups (GDF3/Chow and LacZ/Chow) either in short-term (5 days) or long-term (45 days) (FIG. 6A). High fat diet alone (LacZ/Fat and GDF3/Fat) elevated serum leptin. However, GDF3/Fat mice exhibited much amplified serum leptin level (LacZ/Fat vs. GDF3/Fat 1244±221 vs. 3075±159 pg/mL, P=0.005, 5 days; 1142±231 vs. 2635±153 pg/mL, P=0.017, 45 days). The hyperleptinemic effect of HTTER36 (GDF3) with high fat diet is interpreted as immediate stimulation on adipocytes as suppose to increased fat mass factor, since neither the fat mass nor the adipocyte cell size were sufficiently larger before the onset of obesity by day 5. Thus, HTTER36 (GDF3) with high fat diet strongly induces leptin as a high lipid load signal. However, at the end of equation, the adipogenic activity of HTTER36 (GDF3) overwhelmed the countering lipostatic effect by leptin.

D. Serum Insulin Levels and Blood Glucose Clearance

Methods:

Serum insulin levels were determined by 1-2-3 Rat Insulin ELISA kit (ALPCO Diagnostics, NH) according to the manufacturers' instructions. All measurements were done in triplicates. Raw assay values were converted to leptin or insulin concentrations by standard reference curves and sample dilution factors.

To test for clearance of glucose from the blood, mice were fasted overnight, water ad libitum, prior to administration of the test. In addition, food was not provided during the study. Blood glucose levels were determined by One-touch Ultra Glucometer (Life Scan) with −1 μL blood samples from tail bleed. Mice were orally challenged with 2 g/kg dextrose solution via 22 G gavage feeding. Blood glucose levels just prior to the oral dextrose challenge were measured as the baseline (time 0), and monitored for 2, 5, 15, 30, 60, 120, and 180 minutes thereafter.

Results and Discussion:

Because obesity and type-II diabetes are closely associated metabolic conditions, serum insulin levels and blood glucose clearance were examined in GDF3/Fat, GDF/Chow, LacZ/Fat and LacZ/Chow mice. Blood insulin levels were not different among all groups in long-term treatment (ns, P=0.36 by Krustal-Wallis one-way ANOVA) or between GDF3/Chow and LacZ/Chow mice on day 5 (537±33.5 vs. 582±20.0 pg/mL, ns, P=0.29 by t-test). Short term GDF3/Fat had lower blood insulin than LacZ/Chow (585±12 vs. 699±42; P<0.05). The basal glucose levels of GDF-3/Fat, GDF-3/Chow, LacZ/Fat and LacZ/Chow after overnight fasting were not different (FIGS. 6C and 6D at zero time points, P=0.24 by Krustal-Wallis test). When orally challenged with 2 g/kg dextrose, the glucose was cleared from blood at approximately the same rate for GDF-3 and LacZ groups under the same diet (FIGS. 6C and 6D). The delayed glucose clearance in high fat diet groups (GDF3/Fat and LacZ/Fat) is attributed to their established body overweight. Even though HTTER36 (GDF3) is an adipogenic factor, it does not induce or promote a diabetic condition, which is in agreement with the lack of a correlation between HTTER36 (GDF3) expression and genetically diabetic and obese ob/ob, db/db and tb/tb models (B. A. Witthuln and D. A. Bernlohr, Cytokine, 14:129-135 (2001)). Therefore, HTTER36 (GDF3) can be characterized as a non-diabetic adipogenic factor.

E. PPARγ Expression.

Methods:

Human primary preadipocytes and adipocytes were obtained from Zen-Bio, Inc, mouse 3T3L1 cell line from ATCC. Preadipocytes and undifferentiated 3T3L1 cells were grown in 10 cm cell culture dishes in Zen-Bio Preadipocyte Medium (DMEM/Ham's F12 medium, 15 mM HEPES pH 7.4, 10% fetal bovine serum, 100 U/mL penicillin, 100 U/mL streptomycin, and 0.25 μg/mL amphotericin B). Adipocytes or differentiated 3T3L1 cells were grown in Zen-Bio Adipocyte Medium (DMEM/Ham's F-12 medium, 15 mM HEPES pH7.4, 10% fetal bovine serum, supplemented with 33 μM biotin, 17 μM pantothenate, 100 nM human insulin, 1 μM dexamethasone, 100 U/mL penicillin, 100 U/mL streptomycin, and 0.25 μg/mL amphotericin B). Differentiation of human preadipocytes or 3T3L1 cells was initiated by Zen-Bio Differentiation Medium (Adipocyte Medium supplemented with 0.25 mM isobutylmethylxanthine and 10 μM PPARγ agonist) for 4 days. The initiated cells were allowed to full differentiation in Adipocyte Medium for a week before use.

The cells were treated with or without 500 ng/mL HTTER36 (GDF3) for 48 hours. Total RNA was extracted twice by Trizol method (Invitrogen). 25 ng RNA per test was analyzed by Taqman RT-PCR using mouse PPARγ primer/probe set (primer sequences, 5′-GAATTAGATGACAGTGACTTGGCTATATTTAT-3′ and 5′-TCGATGGGCTTCACGTTCA-3′; probe sequence, 5′-CTCAGTGGAGACCGCCCAGGCTT-3′). Mouse 18s RNA was used as a reference (primer sequences, 5′-CGGCTACCACATCCAAGGAA-3′ and 5′-GCTGGAATTACCGCGGCT-3′; probe sequences, 5′-TGCTGGCACCAGACTTGCCCTC-3′). The abundance of PPARγ RNA was expressed as expression ratio over 18s RNA.

Results and Discussion:

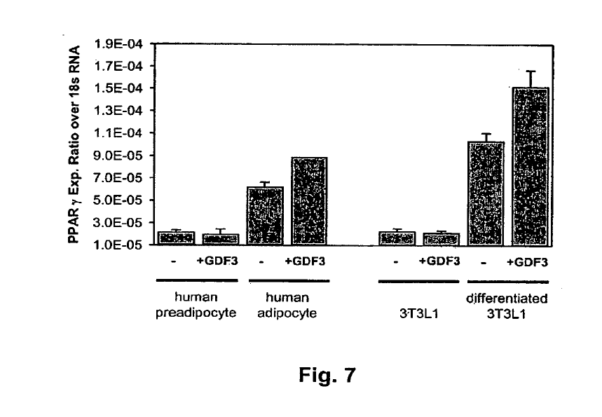

PPARγ expression levels in preadipocytes and adipocytes after HTTER36 (GDF3) treatment were analyzed by Taqman RT-PCR. Human primary adipocytes prepared from human adipose tissue and mouse 3T3L-1 fibroblasts differentiated by insulin, dexamethasone, and thyroxine had high PPARγ levels that were further stimulated by HTTER36 (GDF3) (FIG. 7). Neither human primary preadipocytes nor mouse undifferentiated 3T3L1 cells had PPARγ expression in response to HTTER36 (GDF3).

These results indicate that HTTER36 (GDF3) signaling in mature adipocytes is at least in part mediated by PPARγ. PPARγ is a key regulator of adipocyte differentiation and regulates genes central to lipid metabolism and storage, for example, acetyl-CoA synthetase, aP2, phosphaenol pyruvate carboxykinase, fatty acid transport protein, and lipoprotein lipase. In addition, constitutively active PPARγ has been found to increase adipocyte differentiation and obesity in humans. (Ristow, M. et al., N. Engl. J. Med. 339:953-959 (1998)). Thus, the mediation of HTTER36 (GDF3) signaling, at least in part, by PPARγ indicates that HTTER36 (GDF3) may be useful in the diagnosis and/or treatment of obesity.

Numerous modifications and variations of the present invention are possible in light of the above teachings and, therefore, within the scope of the appended claims, the invention may be practiced otherwise than as particularly described.

1. A method of treating a condition in a subject comprising administering a therapeutically effective amount of an antibody or fragment thereof against GDF3 to said subject, wherein said condition is selected from the group consisting of obesity, excessive appetite, a metabolic syndrome, menopausal associated weight gain, excessive pregnancy weight gain, psychological disorders, weight gain associated with the use of alterations in SNS effects on metabolism, high leptin levels in adolescent females, low perinatal birth weight, and changes in blood pressure.

2. The method of claim 1, wherein said condition is obesity.

3. The method of claim 1, wherein said treating results in weight loss.

4. The method of claim 2, wherein said treating results in reduction of weight gain.

5. The method of claim 1, wherein said metabolic syndrome is selected from the group consisting of insulin resistance, alterations in glucose and lipid metabolism, and visceral obesity.

6. The method of claim 1, wherein said psychological disorder is selected from the group consisting of bipolar disorder and depression.

7. The method of claim 1, wherein said antibody or fragment thereof is selected from the group consisting of a chimeric antibody, single chain antibody, humanized antibody, a Fab fragment, and a product of a Fab expression library.

8. The method of claim 1, wherein said antibody or fragment thereof is monoclonal.

9. The method of claim 1, wherein said antibody fragment thereof is obtained from an animal that has been immunized with a protein selected from the group consisting of:
(a) a protein consisting of amino acid residues 1 to 364 of SEQ ID NO:2;
(b) a protein consisting of amino acid residues 17 to 364 of SEQ ID NO:2;
(c) a protein consisting of amino acid residues 251 to 364 of SEQ ID NO:2;
(d) a protein consisting of the full-length polypeptide encoded by the cDNA contained in ATCC Deposit Number 97349;

10. The method of claim 9, wherein said antibody or fragment thereof is obtained from an animal immunized with protein (a).

11. The method of claim 9, wherein said antibody or fragment thereof is obtained from an animal immunized with protein (b).

12. The method of claim 9, wherein said antibody or fragment thereof is obtained from an animal immunized with protein (c).

13. The method of claim 9, wherein said antibody or fragment thereof is obtained from an animal immunized with protein (d).

14. The method of claim 1, wherein said antibody or fragment thereof is obtained from an animal that has been immunized with a protein at least 70% identical to the sequence of SEQ ID NO: 2, or a fragment thereof, or 95% identical to a protein comprising the polypeptide encoded by the cDNA contained in ATCC Deposit Number 97349, or a fragment thereof.

15. The method of claim 14, wherein said antibody or fragment thereof is obtained from an animal that has been immunized with a fragment is at least 30 consecutive amino-acids.

16. The method of claim 15, wherein said antibody or fragment thereof is obtained from an animal that has been immunized with a fragment is at least 50 consecutive amino-acids.

17. The method of claim 14, wherein said antibody or fragment thereof is obtained from an animal that has been immunized with a protein is at least 90% identical to the sequence of SEQ ID NO:2, or a fragment thereof.

18. The method of claim 17, wherein said antibody or fragment thereof is obtained from an animal that has been immunized with a protein is at least 95% identical to the sequence of SEQ ID NO:2, or a fragment thereof.

19. The method of claim 1, wherein said subject is a human.


SEQUENCE LISTING (9 sequences)

SEQ ID NO: 1 - DNA, 1212 bp, Homo sapiens
  CDS (41)..(1132)
aattcggcacgagcccggtctgacagccactccagaggccatgcttcgtttcttgccagatttggctttcagcttcctgttaattctggctttgggccaggcagtccaatttcaagaatatgtctttctccaatttctgggcttagataaggcgccttcaccccagaagttccaacctgtgccttatatcttgaagaaaattttccaggatcgcgaggcagcagcgaccactggggtctcccgagacttatgctacgtaaaggagctgggcgtccgcgggaatgtacttcgctttctcccagaccaaggtttctttctttacccaaagaaaatttcccaagcttcctcctgcctgcagaagctcctctactttaacctgtctgccatcaaagaaagggaacagttgacattggcccagctgggcctggacttggggcccaattcttactataacctgggaccagagctggaactggctctgttcctggttcaggagcctcatgtgtggggccagaccacccctaagccaggtaaaatgtttgtgttgcggtcagtcccatggccacaaggtgctgttcacttcaacctgctggatgtagctaaggattggaatgacaacccccggaaaaatttcgggttattcctggagatactggtcaaagaagatagagactcaggggtgaattttcagcctgaagacacctgtgccagactaagatgctcccttcatgcttccctgctggtggtgactctcaaccctgatcagtgccacccttctcggaaaaggagagcagccatccctgtccccaagctttcttgtaagaacctctgccaccgtcaccagctattcattaacttccgggacctgggttggcacaagtggatcattgcccccaaggggttcatggcaaattactgccatggagagtgtcccttctcactgaccatctctctcaacaggtccaattatgctttcatgcaagccctgatgcatgccgttgacccagagatcccccaggctgtgtgtatccccaccaagctgtctcccatttccatgctctaccaggacaataatgacaatgtcattctacgacattatgaagacatggtagtcgatgaatgtgggtgtgggtaggatgtcagaaatgggaatagaaggagtgttcttagggtaaacttttaataaaactacctagctggtttatgcccaaa

SEQ ID NO: 2 - protein, 364 aa, Homo sapiens
MLRFLPDLAFSFLLILALGQAVQFQEYVFLQFLGLDKAPSPQKFQPVPYILKKIFQDREAAATTGVSRDLCYVKELGVRGNVLRFLPDQGFFLYPKKISQASSCLQKLLYFNLSAIKEREQLTLAQLGLDLGPNSYYNLGPELELALFLVQEPHVWGQTTPKPGKMFVLRSVPWPQGAVHFNLLDVAKDWNDNPRKNFGLFLEILVKEDRDSGVNFQPEDTCARLRCSLHASLLVVTLNPDQCHPSRKRRAAIPVPKLSCKNLCHRHQLFINFRDLGWHKWIIAPKGFMANYCHGECPFSLTISLNRSNYAFMQALMHAVDPEIPQAVCIPTKLSPISMLYQDNNDNVILRHYEDMVVDECGCG

SEQ ID NO: 3 - DNA, 40 bp, Artificial sequence
  5′ primer with a BamHI site and 18 nt of HTTER36 coding sequence.
gaaaggatccgcagccatccctgtccccaaactttcttgt

SEQ ID NO: 4 - DNA, 40 bp, Artificial sequence
  3′ primer with a HindIII site, a stop codon and 15 nt of HTTER36 sequence.
tccttctattcaagcttctgacatcctacccacacccaca

SEQ ID NO: 5 - DNA, 35 bp, Artificial sequence
  5′ primer with a BamHI site, start codon, and 17 nt of HTTER36 coding sequence.
cagggatccgccatcatgcttcgtttcttgccaga

SEQ ID NO: 6 - DNA, 33 bp, Artificial sequence
  3′ primer with an Asp718 site and 23 nt of HTTER36 3′ sequence.
cttcggtacccatttctgacatcctacccacac

SEQ ID NO: 7 - DNA, 36 bp, Artificial sequence
  5′ primer with a BamHI site followed by an efficient signal for translation, and 18 nt of the gene.
acagcggatccagccaccatgcttcgtttcttgcca

SEQ ID NO: 8 - DNA, 40 bp, Artificial sequence
  3′ primer with a BamHI site and 29 nt of 3′ gene sequence.
tccttcggatcccatttctgacatcctacccacacccaca

SEQ ID NO: 9 - protein, 366 aa, Mus musculus
MQPYQRLLALGFLLLTLPWGQTSEFQDSDLLQFLGLEKAPSPHRFQPVPRVLRKIIRAREAAAASGASQDLCYVKELGVRGNLLQLLPDQGFFLNTQKPFQDGSCLQKVLYFNLSAIKEKAKLTMAQLTLDLGPRSYYNLRPELVVALSVVQDRGVWGRSHPKVGRLLFLRSVPGPQGQLQFNLQGALKDWSSNRLKNLDLHLEILVKEDRYSRVTVQPENPCDPLLRSLHASLLVVTLNPKHCHPSSRKRRAAISVPKGFCRNFCHRHQLFINFQDLGWHKWVIAPKGFMANYCHGECPFSMTTYLNSSNYAFMQALMHMADPKVPKAVCVPTKLSPISMLYQDSDKNVILRHYEDMVVDECGCG